(19) United States
(12) Patent Application Publication
(10) Pub. No.: US 2006/0224234 A1
(43) Pub. Date: Oct. 5, 2006

(54) Drug eluting structurally variable stent

(76) Inventor: Swaminathan Jayaraman, Fremont, CA (US)

Correspondence Address: PAUL D. BIANCO: FLEIT, KAIN, GIBBONS,;GUTMAN, BONGINI, & BIANCO P.L., 21355 EAST DIXIE HIGHWAY, SUITE 115, MIAMI, FL, 33180, US

(21) Appl. No.: 11/399,304

(22) Filed: Apr. 6, 2006

Related U.S. Application Data
(63) Continuation-in-part of application No. 10/696,174, filed on Oct. 29, 2003.
Application No. 10/696,174 is a continuation-in-part of application No. 09/994,253, filed on Nov. 26, 2001, now Pat. No. 6,641,611.
Application No. 10/696,174 is a continuation-in-part of application No. 10/286,805, filed on Nov. 4, 2002, now Pat. No. 6,746,478.
(63) Continuation-in-part of application No. 11/156,992, filed on Jun. 20, 2005.
Application No. 11/156,992 is a continuation-in-part of application No. 09/941,327, filed on Aug. 29, 2001, now Pat. No. 6,908,480.

(51) Int. Cl. A61F 2/90

(52) U.S. Cl. 623/1.16; 623/1.42

(57) ABSTRACT

The present invention provides a stent including a tubular body having a plurality of reservoirs disposed therein and a therapeutic agent located in the reservoirs or located in the reservoirs and on a surface portion of the tubular body, wherein the stent is free of polymeric material. The invention also provides a drug-eluting stent made from the process of providing a polymer-free stent body having a plurality of reservoirs disposed therein, diluting a therapeutic agent in a polymer-free solvent to form an agent-solvent mixture, coating the stent with the agent-solvent mixture, and allowing the solvent to dissipate from the stent thereby leaving the agent disposed on the stent.

### CROSS-REFERENCE TO RELATED APPLICATION

This application is a continuation-in-part of U.S. patent application Ser. No. 10/696,174 filed on Oct. 29, 2003. U.S. patent application Ser. No. 10/696,174 is a continuation-in-part of U.S. patent application Ser. No. 09/994,253 filed on Nov. 26, 2001, now U.S. Pat. No. 6,641,611. U.S. patent application Ser. No. 10/696,174 is also a continuation-in-part of U.S. patent application Ser. No. 10/286,805 filed on Nov. 4, 2002, now U.S. Pat. No. 6,746,478. The present application is also a continuation-in-part of U.S. patent application Ser. No. 11/156,992 filed on Jun. 20, 2005. U.S. patent application Ser. No. 11/156,992 is a continuation-in-part of U.S. patent application Ser. No. 09/941,327 filed on Aug. 29, 2001, now U.S. Pat. No. 6,908,480. The contents of the above-mentioned patent documents are herein incorporated by reference in their entirety.

### FIELD OF THE INVENTION

The present invention relates to implants used to support arterial and venous conduits in the human body. More particularly, the invention provides a tubular stent having a non-uniform structure along its longitudinal length and has reservoirs therein to carry a therapeutic agent.

### BACKGROUND OF THE INVENTION

Intravascular diseases, such as stenosis, may be treated by non-invasive techniques such as percutaneous transluminal angioplasty (PTA) and percutaneous transluminal coronary angioplasty (PTCA). These therapeutic techniques are well known in the art and typically involve use of a guide wire and a balloon catheter, possibly in combination with other intravascular devices, to open the restriction in the vessel. However, vascular restrictions that have been dilated do not always remain open. Restenosis occurs causing the vessel to restrict again.

The concept of restenosis or hyperproliferative vascular disease is now being more clearly understood then it was a couple of years ago. The distinctive feature of restenosis is its diverse histopathology. Histologically, restenosis is characterized by a diffuses, concentric, fibrous expansion of the graft arterial intima, termed neointimal hyperplasia. Growth of this lesion, which is often accompanied by fragmentation of the internal elastic lamina, results in progressive vascular occlusion and is seen as a reduction in lumen cross-sectional area in histological sections or upon angiography or other intravascular techniques. Neointimal hyperplasia, together with constrictive vascular remodeling, eventually culminates in complete arterial occlusion.

Restenosis was simply thought to be a response of the vascular smooth muscle cells upon injury. There is now information available to demonstrate that the restenosis process is different in every individual depending on the underlying conditions that constitute the vascular disease. These underlying conditions can be classified as diabetic, non-diabetic, small vessels, larger vessels, complex diseases, pro-atherogenic vessels, etc. Depending on the various mechanisms of the underlying complications, the restenotic process is different and various drugs and combination of drugs can used to treat or prevent a specific disease process of the vascular disease.

A stent is a type of endovascular implant, usually generally tubular in shape, which is expandable to be permanently inserted into the blood vessel to provide mechanical support to the vessel and to maintain or re-establish a flow channel during or following angioplasty. The support structure of the stent is designed to prevent early collapse of a vessel that has been weakened and damaged by angioplasty.

There are generally four classes of stents employed in the prior art. First, coil stents are made from a single wire. The wire is bent in various ways and formed into a stent. Examples of this type of stent are those shown in U.S. Pat. Nos. 4,969,458; 4,681,110 and 5,824,056. Second, slotted tube stents are laser cut using a tube of either stainless steel, nickel/titanium alloy (NITINOL), titanium or any other suitable materials. These designs are preprogrammed into a machine language, and a laser is used to cut in accordance with the programs. These stents have a uniform design and a uniform thickness from the beginning to the end of the stent. In other words, the same segment is repeated from one end of the stent to the other. Examples of this type of stent are described in U.S. Pat. Nos. 4,733,665; 4,739,762; 4,776,337 and 4,793,348.

The third class of stents is self-expanding stents which are usually braided or knitted with multiple wire filaments such that they have a lower profile when stretched and they expand from a lower profile to a higher profile when unconstrained in the body. These stents are called self-expanding stents and are described in U.S. Pat. No. 4,655,771. Fourth, hybrid stents are similar to slotted tube stents except that they do not have a closed cell structure but have an open cellular structure with flexible interconnections between each segment of the design. These interconnections provide the flexibility while the segments provide the radial strength and other important properties of the stent. Examples of this stent are described in U.S. Pat. Nos. 5,514,154; 5,562,728; 5,649,952 and 5,725,572.

Many implants, including balloons and stents, have a coating. For example, U.S. Pat. No. 5,759,174 describes a balloon that has a radiopaque segment attached to one of the longitudinal ends of the balloon. When the balloon is inflated, the stent is pressed against the ends of the artery and this indicates the center portion of the dilated stenosis. The external radiopaque marker band is typically made from a dense radiopaque metal such as tantalum, gold, platinum or an alloy of those dense metals.

U.S. Pat. No. 5,725,572 describes gold plating on the ends of a stent such that the gold plating marks two bands at the ends of a stent. The patentee mentions that the limitation of gold coating is the stiffening of the stent surface. Hence, the gold plating is done only at the ends where the stiffening does not significantly alter the properties of the stent. Also described is another embodiment where only the exterior of the stent is coated with a radiopaque material.

U.S. Pat. No. 5,919,126 describes a stent where the body of the stent is formed from a non-radioactive structural material, a radiopaque material coats the body and a beta emitting radioisotope ion is implanted into the radiopaque material.

U.S. Pat. No. 5,824,056 describes an implantable medical device formed from a drawn refractory metal having an improved biocompatible surface. The method by which the device is made includes coating a refractory metal article with platinum by a physical vapor deposition process and subjecting the coated article to drawing in a diamond die. The drawn article can be incorporated into an implanted medical device without removing the deposited material.

U.S. Pat. No. 5,824,077 describes a stent which is formed of multiple filaments arranged in two sets of oppositely directed helical windings interwoven with each other in a braided configuration. Each of the filaments is a composite including a central core and a case surrounding the core. The core is formed of a radiopaque material while the outer casing is made of a relatively resilient material, e.g., a cobalt chromium based alloy. Alternative composite filaments described in the patent employ an intermediate barrier layer between the case and the core, a biocompatible cover layer surrounding the case, and a radiopaque case surrounding the central core.

U.S. Pat. No. 5,871,437 describes a non-radioactive metallic stent which is coated with a biodegradable or non-biodegradable thin coating of less than about 100 microns in thickness which is selected to avoid provoking any foreign body reaction. This coating contains a radioactive source emitting Beta particles with an activity level of approximately one micro curie and on top of this layer is an anticoagulant substance to inhibit early thrombus formation.

U.S. Pat. No. 6,099,561 describes a stent having a biocompatible metal hollow tube constituting a base layer having a multiplicity of openings through an open ended tubular wall thereof, the tube constituting a single member from which the entire stent is fabricated. A thin tightly adherent intermediate layer of noble metal overlies the entire exposed surface area of the tube including edges of the openings as well as exterior and interior surfaces and ends of the wall. A third outermost ceramic like layer composed of an oxide, hydroxide or nitrate of a noble metal is formed atop and in adherent relation to an intermediate layer.

U.S. Pat. No. 5,722,984 describes a stent which has an antithrombogenic property and contains an embedded radioisotope that makes the coating material radioactive. Other relevant patents that describe the coating technology or coating properties include U.S. Pat. Nos. 5,818,893; 5,980,974; 5,700,286; 5,858,468; 5,650,202 and 5,696,714.

The prior art also discloses many examples of therapeutic coatings that have been applied to intravascular devices, such as stents. The objective behind applying the therapeutic coating is to either mediate or suppress a tissue response at the site of implantation. For example in intravascular situations, one of the obvious outcomes of implanting a foreign body is for an intense reaction at the site of implantation. This intense reaction can result from either the implantation itself or the stresses generated after implantation. Due to the reaction, there is an obvious interaction by the vessel wall to compensate for this injury by producing a host of tissue related responses that is generally called “healing due to injury.” It is this healing process that the therapeutic coating attempts to mediate, suppress, or lessen. In some instances, this healing process is excessive in which it occludes the entire lumen providing for no blood flow in the vessel. This reoccluded vessel is also called a restenotic vessel.

Therapeutic coatings can affect the vascular disease or disease process in different ways. For example, depending upon the kind of therapeutic agent used, the various cellular levels of mechanisms are tackled. Some of the therapeutic agents act on the growth factors that are generated at the site of implantation or intervention of the vessel. Some other therapeutic agents act on the tissues and suppress the proliferative response of the tissues. Others act on the collagen matrix that comprises the bulk of the smooth muscle cells. Some examples of prior art relating to therapeutic coatings follow.

U.S. Pat. No. 5,283,257 issued to Gregory et al. provides a method of preventing or treating hyperproliferative vascular disease in a mammal by administering an amount of mycophenolic acid effective to inhibit intimal thickening. This drug can be delivered either after angioplasty or via a vascular stent that is impregnated with mycophenolic acid.

U.S. Pat. No. 5,288,711 issued to Mitchell et al. provides a method of preventing or treating hyperproliferative vascular disease in a mammal by administering an antiproliferative effective amount of a combination of rapamycin and heparin. This combination can be delivered either after angioplasty or via a vascular stent that is impregnated with the combination.

U.S. Pat. Nos. 5,516,781 and 5,646,160 issued to Morris et al. disclose a method of preventing or treating hyperproliferative vascular disease in a mammal by administering an antiproliferative effective amount of rapamycin alone or in combination with mycophenolic acid. The rapamycin or rapamycin/mycophenolic acid combination can be delivered via a vascular stent.

U.S. Pat. No. 5,519,042 issued to Morris et al. teaches a method of preventing or treating hyperproliferative vascular disease in a mammal consists of administering to a mammal an effective amount of carboxyamide compounds. This can also be delivered intravascularly via a vascular stent.

U.S. Pat. No. 5,646,160 issued to Morris et al. provides a method of preventing or treating hyperproliferative vascular disease in a mammal by administering an antiproliferative effective amount of rapamycin alone or in combination with mycophenolic acid. This can be delivered intravascularly via a vascular stent.

Each of the above-identified patents utilizes an immunosuppressive agent. Since the mid 1980's, many new small molecular weight molecules of natural product, semi-synthetic or totally synthetic origin have been identified and developed for the control of graft rejection. These include mizoribine, deoxyspergualin, cyclosporine, FK 506, mycophenolic acid (and its prodrug form as mycophenolate mofetil), rapamycin, and brequinar sodium. The mechanisms of some of these agents will now be briefly summarized.

Both cyclosporine and FK 506 suppress T-cell activation by impeding the transcription of selected cytokine genes in T cells. Neither has any known direct effects on B cells. The suppression of interleukin 2 (IL-2) synthesis is an especially important effect of these two agents, because this cytokine is required for T cells to progress from initial activation to DNA synthesis. Both cyclosporine A and FK 506 bind to cytoplasmic proteins. It has been recently proposed that cyclosporine A and FK 506 are bifunctional: one segment of the immunosuppressant molecule is responsible for binding to the rotamase and, once bound, a separate part of the molecule interacts with a cytoplasmic phosphatase (calcineurin) and causes the phosphatase to become inactive or have altered specificity. Unlike all previously developed immunosuppressants and even the most recent xenobiotic immunosuppressants, FK 506 is the only compound in the history of immunosuppressive drug development that is the product of a drug discovery program designed specifically to identify an improved molecule for the control of allograft rejection. Every other past and “new” immunosuppressive xenobiotic drug is the unanticipated result of drug discovery programs organized to identify lead compounds for anticancer, anti-inflammatory, or antibiotic therapy.

Neither cyclosporine, FK 506, rapamycin nor other immunosuppressants are the product of evolutionary pressures that led to our current use of them as immunosuppressants. The agents are fungal (cyclosporine A) or bacterial (FK 506, rapamycin) metabolites that suppress lymphocyte proliferation purely through coincidental molecular interactions. Therefore, as our ability to design drugs that perform specific intravascular functions increases, there should be a reciprocal decrease in the severity of their adverse effects.

There is a need for safer versions of cyclosporine, FK 506, rapamycin and mycophenolic acid as well as for analogues with higher immunosuppressive efficacy. Because of their toxicities, these agents cannot be used at maximally immunosuppressive doses.

Another significant issue that complicates the delivery of relatively high dosage of the agents is the relatively narrow therapeutic window. This narrow window of therapeutic vs. toxicity restricts most of these agents to be used as monotherapy for intravascular delivery.

Rapamycin, for example, inhibits the IL-2 induced proliferation of specific IL-2 responsive cell lines, but neither cyclosporine nor other drugs can suppress this response. Because rapamycin acts late in the activation sequence of T cells, it also effectively inhibits T cells inactivated by a recently described calcium independent pathway. Thus, T cells stimulated through this alternative route are insensitive to suppression by cyclosporine A and FK 506, but rapamycin inhibits their proliferation only.

The toxicity profile of rapamycin resembles cyclosporine A and FK 506. Rapamycin is associated with weight loss in several species, and treatment with high doses of rapamycin causes diabetes in rats, but not in nonhuman primates. Initial animal data suggests that rapamycin may be less nephrotoxic than cyclosporine A, but its effects on kidneys with impaired function have not been evaluated. Rapamycin at highly effective therapeutic doses is highly toxic and its usage is recommended along with a combination of other immunosuppressants. The combination with cyclosporine A results in a significant increase in the therapeutic level in blood when compared with monotherapy. A lower dosage of the combination is more effective than a higher dosage of monotherapy. The dosage of rapamycin could be reduced nine fold and cyclosporine A could be reduced five fold when these agents are used in combination. In addition, the combination is also not toxic. In fact, the U.S. FDA has approved the usage of rapamycin for transplantation and allograft rejection only upon combination therapy with cyclosporine.

In summary, the problems associated with immunosuppressive agents include, narrow therapeutic window, toxicity window, inefficacy of agents, and dosage related toxicity. In order to overcome these problems, combination therapy involving two agents has been used with success. It has been surprisingly found that the benefits of combined immunosuppression with rapamycin and cyclosporine A have a very synergistic approach towards cellular growth and retardation. Studies have shown that suppression of heart graft rejection in nonhuman primates is especially effective when rapamycin is combined with cyclosporine A. The immunosuppressive efficacy of combined therapy is superior to treatment with either agent alone; this effect is not caused by the elevation of cyclosporine A blood levels by co-administration of rapamycin. The combination treatment with rapamycin and cyclosporine A does not cause nephrotoxicity. The distinct sites of immunosuppressive action of cyclosporine A and rapamycin (cyclosporine A acts on the calcium dependent and rapamycin acts on the calcium independent pathway) and their relatively non-overlapping toxicities will enable this combination to be used intravascularly to prevent cellular growth at the site of injury inside the blood vessel after angioplasty.

Several scientific and technical publications mention the “surprisingly” “synergistic” effect of rapamycin and cyclosporine A. For example, Schuurman et al. in Transplantation Vol 64, 32-35, No. 1, Jul. 15, 1997 describe SDZ-RAD, a new rapamycin derivative that has a synergism with cyclosporine. They conclude that both the drugs show synergism in immunosuppression, both in vitro and in vivo. The drugs are proposed to have a promising combinatorial therapy in allotransplantation.

Schuler et al. in Transplantation Vol 64, 36-42, No. 1, Jul. 15, 1997 report that the drug rapamycin by itself has a very narrow therapeutic window, thus decreasing its clinical efficacy. They reported that in combination with cyclosporine A, the drugs act in a synergistic manner. This synergism, if proven in humans, offers the chance to increase the efficacy of the immunosuppressive regimen by combining the two drugs, with the prospect of mitigating their respective side effects. The authors also propose that they believe that the increased immunosuppressive efficacy of a drug combination composed of cyclosporine A and rapamycin, combined with the ability of rapamycin to prevent VSMC proliferation, bears the potential for improving the prospects for long term graft acceptance.

Morris et al. in Transplantation Proceedings, Vol 23, No. 1 (February), 1991: pp 521-524 describe the synergistic activity of cyclosporine A and rapamycin for the suppression of alloimmune reactions in vivo.

Schuurman et al. in Transplantation Vol 69, 737-742, No. 5, Mar. 15, 2000 describe the oral efficacy of the macrolide immunosuppressant rapamycin and of cyclosporine microemulsion in cynomalgus monkey kidney allotransplantation. The authors describe the synergistic activity of both these combinations and explain the possible explanation for failure of rapamycin monotherapy to ensure long term survival in this animal model might be the different mode of action of the compound when compared to cyclosporine. Cyclosporine acts very early in the chain of events that lead to a T-cell immune response. It blocks the antigen-driven activation of T cells, inhibiting the production of early lymphokines by interfering with the intracellular signal that emanates from the T-cell receptor upon recognition of antigen. Rapamycin acts rather late after T cell activation. The authors conclude that drugs like rapamycin need to be combined with immunosuppressants like cyclosporine to inhibit the early T-cell activation event and thus prevent an inflammatory response.

Hausen et al. in Transplantation Vol 69, 488-496, No. 4, Feb. 27, 2000 describe the prevention of acute allograft rejection in nonhuman primate lung transplant recipients. The authors mention that fixed dose studies using monotherapy with either high dose cyclosporine A or a high dose rapamycin did not prevent early acute allograft rejection, but monotherapy with either drug was well tolerated. The fixed doses of the drugs were used in combination, but this led to 5 fold increase in rapamycin levels compared to levels in monkeys treated with rapamycin alone. To compensate for this adverse drug-drug interaction, concentration controlled trials were designed to lower rapamycin levels and cyclosporine A levels considerably when both the drugs were used together. This specimen suppressed rejection successfully.

Martin et al. in the Journal of Immunology in 1995 published a paper “Synergistic Effect of Rapamycin and cyclosporine A in the Treatment of Experimental Autoimmune Uveoretinitis”. The authors conclude that immunosuppressive drugs currently available for the treatment of autoimmune diseases display a narrow therapeutic window between efficacy and toxic side effects. The use of combination of drugs that have a synergistic effect may expand this window and reduce the risk of toxicity. The studies demonstrated synergistic relationship between rapamycin and cyclosporine A and the combination allows the use of reduced doses of each drug to achieve a therapeutic effect. The use of lower doses may also reduce the toxicity of these drugs for the treatment of autoimmune uveitis.

Henderson et al. in Immunology 1991, 73: 316-321 compare the effects of rapamycin and cyclosporine A on the IL-2 production. While rapamycin did not have any effect on the IL-2 gene expression, cyclosporine A did have an effect on the IL-2 gene expression. This shows that the two drugs have a completely different pathway of action.

Hausen et al. in Transplantation Vol 67, 956-962, No. 7, Apr. 15, 1999 published the report of co administration of Neural (cyclosporine A) and the novel rapamycin analog (SDZ-RAD), to rat lung allograft recipients. They mention the synergistic effect of the two compounds—cyclosporine A inhibits early events after T-cell activation, rapamycin affects growth factor driven cell proliferation. Simultaneous administration of cyclosporine A and rapamycin has shown to result in significant increases in rapamycin trough (levels of the drug in blood) when compared with monotherapy. In preclinical and clinical trials, the immunosuppressive strategies have been designed to take advantage of the synergistic immunosuppressive activities of cyclosporine A given in combination with rapamycin. In addition to immunosuppressive synergism, a significant pharmacokinetic interaction after simultaneous, oral administration of cyclosporine A and rapamycin has been found in animal studies.

Whiting et al. in Transplantation Vol 52, 203-208, No. 2, August 1991 describe the toxicity of rapamycin in a comparative and combination study with cyclosporine at immunotherapeutic dosage in the rat.

Yizheng Tu et al. in Transplantation Vol 59, 177-183, No. 2 Jan. 27, 1995 published a paper on the synergistic effects of cyclosporine, Siolimus (rapamycin) and Brequinar on heart allograft survival in mice.

Yakimets et al. in Transplantation Vol 56, 1293-1298, No. 6, December 1993 published the “Prolongation of Canine Pancreatic Islet Allograft Survival with Combined rapamycin and cyclosporine Therapy at Low Doses”.

Vathsala et al. in Transplantation Vol 49, 463-472, No. 2, February 1990 published the “Analysis of the interactions of Immunosuppressive drugs with cyclosporine in inhibiting DNA proliferation”.

The combination of rapamycin and cyclosporine A has been delivered for the treatment of many diseases. For example, U.S. Pat. No. 5,100,899 issued to Calne provides a method of inhibiting organ or tissue transplant rejection in a mammal. The method includes administering to the mammal a transplant rejection inhibiting amount of rapamycin. Also disclosed is a method of inhibiting organ or tissue transplant rejection in a mammal that includes administering (a) an amount of rapamycin in combination with (b) an amount of one or more other chemotherapeutic agents for inhibiting transplant rejection, e.g., azathiprine, corticosteroids, cyclosporine and FK 506. The amounts of (a) and (b) together are effective to inhibit transplant rejection and to maintain inhibition of transplant rejection.

U.S. Pat. No. 5,212,155 issued to Calne et al. claims a combination of rapamycin and cyclosporine that is effective to inhibit transplant rejection.

U.S. Pat. No. 5,308,847 issued to Calne describes a combination of rapamycin and axathioprine to inhibit transplant rejection.

U.S. Pat. No. 5,403,833 issued to Calne et al. described a combination of rapamycin and a corticosteroid to inhibit transplant rejection.

U.S. Pat. No. 5,461,058 issued to Calne describes a combination of rapamycin and FK 506 to inhibit transplant rejection.

U.S. Pat. No. 6,455,518 describes a synergistic combination of IL-2 transcription inhibitor (e.g., cyclosporine A) and a derivative of rapamycin, which is useful in the treatment and prevention of transplant rejection and also certain autoimmune and inflammatory diseases, together with novel pharmaceutical compositions comprising an IL-2 transcription inhibitor in combination with rapamycin.

U.S. Pat. No. 6,239,124 issued to Zenke et al. also describes a synergistic combination of IL-2 transcription inhibitor and rapamycin which is useful in the treatment and prevention of transplant rejection and also certain autoimmune and inflammatory diseases, together with novel pharmaceutical compositions comprising an IL-2 transcription inhibitor in combination with rapamycin.

U.S. Pat. No. 6,051,596 issued to Badger describes a pharmaceutical composition containing a non-specific suppressor cell inducing compound and cyclosporine A in a pharmaceutically acceptable carrier. The patent also discloses a method of inducing an immunosuppressive effect in a mammal, which comprises administering an effective dose of the non-specific suppressor cell inducing compound and cyclosporine A to such mammal.

U.S. Pat. No. 6,046,328 issued to Schonharting et al. describes the preparation and combination of a Xanthine along with cyclosporine A or FK 506.

U.S. Pat. Nos. 5,286,730 and 5,286,731 issued to Caufield et al. describe the combination of rapamycin and cyclosporine A useful for treating skin diseases, and the delivery of the above compounds orally, parentally, intranasally, intrabronchially, topically, transdermally, or rectally.

Published International Application No. WO 98/18468 describes the synergistic composition comprising rapamycin and Calcitriol.

U.S. Pat. Nos. 5,624,946 and 5,688,824 issued to Williams et al. describe the use of Leflunomide to control and reverse chronic allograft rejection.

U.S. Pat. No. 5,496,832 issued to Armstrong et al. provides a method of treating cardiac inflammatory disease which comprises administering rapamycin orally, parenterally, intravascularly, intranasally, intrabronchially, transdermally or rectally.

Although drug-eluting or drug coated stents are widely used for treatment of occlusive vascular diseases, there are risks associated with stents that use polymeric material to carry or disperse therapeutic agents. In a recent study, drug-eluting stents were found to cause allergic reactions that may have serious consequences. Some symptoms experienced by patients having allergic reactions to stents include rash, difficulty breathing, hives, itching, and fever. The study concluded that the polymeric coating on the stents was the most probable cause of the allergic reactions.

While some of the above mentioned documents have disclosed various stents and stent coatings, there is a need for a polymer-free stent that has both good flexibility and radial strength together with the ability to retain a therapeutic agent.

### SUMMARY OF THE INVENTION

The present invention describes a new type of stent having multiple designs of structurally variable configuration along the longitudinal length of the stent. The stent has one pattern at both ends of the stent to provide optimal flexibility and a different pattern at the mid-portion of the stent to provide optimal radial strength. Alternatively, the stent has one pattern at each end, a different pattern at its mid-portion, and a third pattern in-between the mid-portion and each end. The stent has both closed cell and open cell configuration along its longitudinal length and the closed cells and open cells are interlinked with either straight or wavy configurations in a single stent.

An exemplary pattern of the stent contains at least three different configurations selected from an open cell design, a closed cell design, a straight interlink or articulation and one wavy interlink or articulation along a variable thickness of connecting stents. Also, reservoirs or wells may be disposed on at least one of the end portions and mid-portion of the stent. Because of the variable thickness of the portions of the stent and the reservoirs disposed therein, the amount of therapeutic agent loaded on the stent is varied along the length of the stent with various release characteristics.

The structurally variable stent of this invention may have a stainless steel or nickel/titanium alloy (NITINOL) base material. The stent may include two layers of coating together not exceeding ten microns in depth. One layer is an undercoat in direct contact with the base metal both on the inside and outside surface of the base metal. The topmost layer is in contact with the blood. Both the undercoat and top coat are of the same material such as metallic, biological, synthetic material, or polymeric material. Alternatively, the stent may be free of any polymeric material. The polymer-free stent may include a layer of one or more therapeutic agents and a top coat thereon, or the polymer-free stent may include one or more therapeutic agents disposed in reservoirs with a top coat thereon.

In accordance with one aspect of the present invention, a method for treating a vascular disease of a patient with an intravascular implant is provided. The method includes identifying a disease process in the pathology of the vascular disease and selecting a first agent to treat or prevent the vascular disease. The method also includes coating at least a portion of the intravascular implant with a therapeutically effective amount of the first agent and implanting the intravascular implant in the patient.

The present invention also describes a method of making a drug-eluting or drug coated structurally variable stent for treating a vascular disease. The method includes identifying a disease process in the pathology of a vascular disease of a patient and selecting a first agent to treat or prevent the vascular disease. The method also includes coating at least a portion of and/or disposing in wells of the intravascular implant a therapeutically effective amount of the first agent.

The disease process may be identified using an angiogram, fluoroscopy, CT scan, MRI, intravascular MRI, lesion temperature determination, genetic determination, or combination thereof. The disease process may include acute myocardial infarction, thrombotic lesions, unstable angina, fibrotic disease, total occlusion, hyperproliferative vascular disease, vulnerable plaque, diabetic vascular diffused disease, or a combination thereof. The first agent may act on a calcium independent cellular pathway or may be a macrolide immunosuppressant, like rapamycin.

The method may further include selecting a second agent to treat or prevent the vascular disease and coating at least a portion of and/or disposing in wells of the intravascular implant a therapeutically effective amount of the second agent. The second agent may be an anti-inflammatory agent, non-proliferative agent, anti-coagulant, anti-platelet agent, Tyrosine Kinase inhibitor, anti-infective agent, anti-tumor agent, anti-leukemic agent, or a combination thereof.

Moreover, the method may include coating at least a portion of and/or disposing over the wells of the intravascular implant a top coat. The top coat may include a bioabsorbable polymer, poly-α hydroxy acids, polyglycols, polytyrosine carbonates, starch, gelatins, cellulose and combinations thereof. The therapeutically effective amount of the first and/or second agent may be dispersed within the top coat.

Furthermore, the intravascular implant may be, but is not limited to, a balloon catheter, stent, stent graft, stent preform, drug delivery catheter, atherectomy device, filter, scaffolding device, anastomotic clip, anastomotic bridge, suture material, metallic or non-metallic wire, embolic coil or a combination thereof. The intravascular implant may include a primer layer upon which the therapeutic agent(s) is applied. The primer layer may be made of a non-polymeric or polymeric material. The primer layer may be bioabsorbable or biostable.

A more detailed explanation of the invention is provided in the following description and claims, and is illustrated in the accompanying drawings.

### BRIEF DESCRIPTION OF THE DRAWINGS

The invention can be best understood by those having ordinary skill in the art by reference to the following detailed description when considered in conjunction with the accompanying drawings in which:

FIG. 1 shows a closed cell design of a stent;

FIG. 2 shows a closed cell design of a stent interconnected by a straight bridge;

FIG. 3 shows an exterior design of a closed cell stent;

FIG. 4 shows a design of an open cell stent with a radiopaque coating on one section of the stent;

FIG. 5 shows a design of a coil stent;

FIG. 6 shows a design of a structurally variable stent having an open cell design on the ends and a closed cell design at the center of the stent;

FIG. 7 shows a design of a structurally variable stent with variable thickness of the open and closed cell design;

FIG. 8 shows a design of a structurally variable stent with open cell at the ends and closed cell at the mid-portion and alternating articulations between both the open and closed cell;

FIG. 9 shows a design of a structurally variable stent with both open and closed cell design and the articulations at the end of the closed cell design is an S-shape rather than a W-shape;

FIG. 10 shows a design of a structurally variable stent with both open and closed cell design and alternating articulations at various sections of the stent;

FIG. 11 shows a design of a structurally variable stent with an open cell design at the ends with multiple S-shapes and a straight articulating member and closed cell design and the mid-portion with a complex plus sign articulation;

FIG. 12 shows a design of a structurally variable stent with a circle at a mid-portion of the open cell design;

FIG. 13 shows a design of a structurally variable stent with different wall thickness along the length of the stent;

FIG. 14 shows a cross sectional view of a portion of the structurally variable stent including two coating layer;

FIG. 15 shows a partial view of a section of stent including a plurality of reservoirs or wells therein;

FIG. 16 shows a section view of the partial section of FIG. 15;

FIGS. 17A-17F show exemplary cross-sectional reservoir configurations of a stent;

FIG. 18 shows a design of a structurally variable stent with both open and closed cell designs including reservoirs at various sections of the stent;

FIG. 19 is a photograph showing a stent having reservoirs disposed therein;

FIG. 20 is a photograph showing a close-up of the reservoirs of FIG. 19;

FIG. 21 is a cross-sectional view of a stent having various reservoir and tunnel configurations;

FIG. 22 is a cross-sectional view of double-walled stent having various reservoirs and reservoir openings;

FIG. 23 shows a stent preform of the present invention;

FIG. 24 shows a cross-sectional view of a stent preform;

FIG. 25 shows a cross-sectional view through another embodiment of a stent preform;

FIG. 26 shows yet another embodiment of the stent preform including a lubricious lining;

FIG. 27 shows still another embodiment of the stent preform using a tape as an outer sheathing;

FIG. 28 shows a braided stent formed from a stent preform

FIG. 29 is a cross-sectional view of a stent preform having a plurality of drug reservoirs therein;

FIG. 30 shows a cross-sectional view of a stent preform having various drug reservoirs and tunnels therein;

FIG. 31 shows the chemical structures of various macrocyclic immunosuppressants;

FIG. 32 shows a schematic of possible sites of action of cyclosporine A, FK 506, rapamycin, mizoribine, mycophenolic acid, brequinar sodium, and deoxyspergualin on T cell activation by calcium dependent or independent pathways. Certain immunosuppressants also affect B cells and their possible sites of action are also shown;

FIG. 33 shows a schematic of the effects of cyclosporine A, FK 506, rapamycin, mizoribine, mycophenolic acid, and brequinar sodium on the biochemistry of T cell activation;

FIG. 34 shows a graph comparing the effects of cyclosporine A alone (white bars), rapamycin alone (hatched bars), and the combination of cyclosporine A and rapamycin (black bars) on the proliferative response of cells;

FIG. 35 shows an isobologram analysis of a combination of cyclosporine A and rapamycin. The line drawn from 1 to 1 is the line of unity. Combinations that fall below this unity line are synergistic, on the line additive, and above the line antagonistic. The units on the X-axis are Fractional Inhibitory Concentration (FIC) of rapamycin and the units on the Y-axis are FIC of cyclosporine A;

FIG. 36 shows an isobologram analysis of a combination of cyclosporine A and rapamycin. The units on the X-axis are FIC of rapamycin and the units on the Y-axis are FIC of cyclosporine A. The combination at which the maximum proliferative response was inhibited was used to plot the synergistic interaction between the two;

FIG. 37 shows a graph illustrating the amount of proliferation of vascular smooth cells based on different treatments;

FIG. 38 is a bar graph showing the effect of certain therapeutic agents on SMC proliferation;

FIG. 39 is a bar graph illustrating the effect of certain therapeutic agents on SMC migration;

FIG. 40 illustrates the effect of certain therapeutic agents on alphα-SM actin expression S- and R-SMCs; and

FIG. 41 illustrates the effect of certain therapeutic agents on S100A4 expression in S- and R-SMCs.

### DETAILED DESCRIPTION OF THE INVENTION

The present invention provides a tubular self support structure composed of a biocompatible material which can be used as a stent to support arterial and venous conduits in the human body. The stent can include one or more patterns of interconnected lattice works which can be connected by strut members. The patterns can be in the form of a “closed” cell or “open” cell design, wherein “closed cell” and “open cell” are terms of art that a person of ordinary skill in the art would readily understand and appreciate what is covered by the recitation of “closed cell” and “open cell.” Specifically, an open cell stent is defined as a stent that has circumferential sets of strut members with most of the curved sections that are not connected by a longitudinal connecting link to an adjacent circumferential set of struts. A closed cell stent has every curved section of every circumferential set of strut members, except at the distal and proximal end of the stent, attached to a longitudinal connecting link. The definitions of “open cell” and “closed cell” are provided, for example, in U.S. Pat. No. 6,540,774, to Fischell et al, entitled “Ultraflexible Open Cell Stent.”

The intravascular implants of the present invention (e.g. stents or stent preforms) also include one or more reservoirs or tunnels disposed therein. One or more therapeutic agents may be placed in the reservoirs and/or on the surface of the implant. Because of the variable thickness of the portions of the stent and the reservoirs disposed therein, the amount of therapeutic agent loaded on the stent is varied along the length of the stent with various release characteristics. The stent may include two layers of coating together not exceeding ten microns in depth. One layer is an undercoat in direct contact with the base metal both on the inside and outside surface of the base metal. The topmost layer is in contact with the blood. Both the undercoat and top coat are of the same material such as metallic, biological, synthetic material, or polymeric material. Alternatively, the stent may be free of any polymeric material. The polymer-free stent may include a layer of one or more therapeutic agents and a top coat thereon, or the polymer-free stent may include one or more therapeutic agents disposed in reservoirs with a top coat thereon.

Structurally Variable Stents

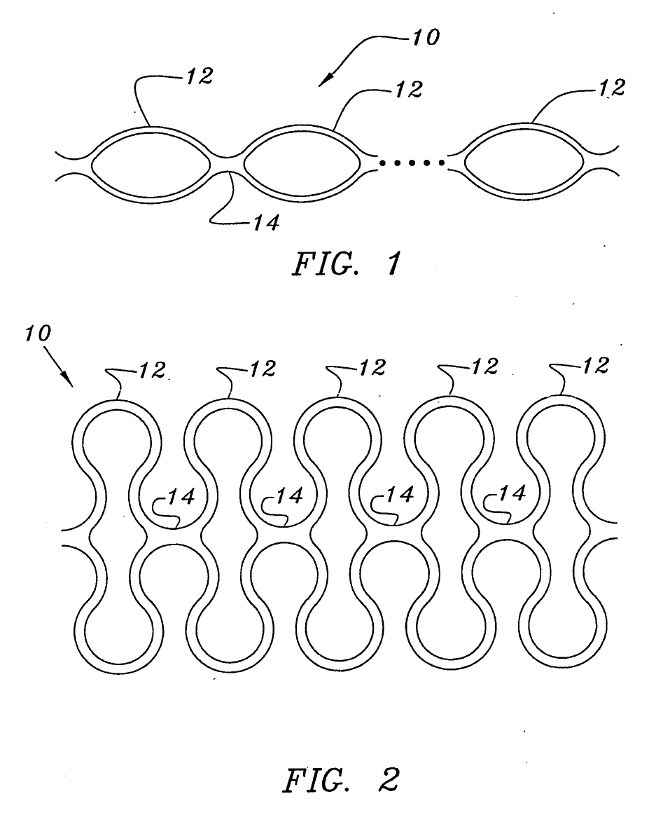

Referring now to the drawing figures in which like reference designators refer to like elements, there is shown in FIG. 1, a longitudinal sectional view of a stent 10 of the present invention. The stent 10 includes a series of cells 12 which are longitudinal connected in series, where the cells 12 are interconnected by bridge or strut member 14. The longitudinal serial connections of the cells 12 define the stent as a “closed” cell stent.

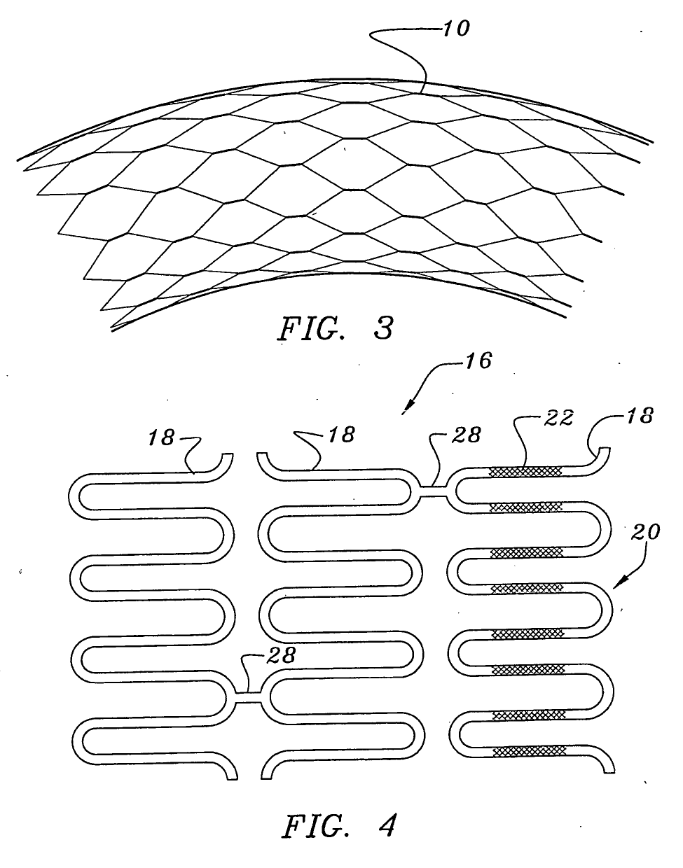

The cells 12 are depicted as having a substantially elliptical shape. However, as shown in FIG. 2, the cells 12 can have a more complex shape. The exterior look of such a stent 10 is provided in FIG. 3.

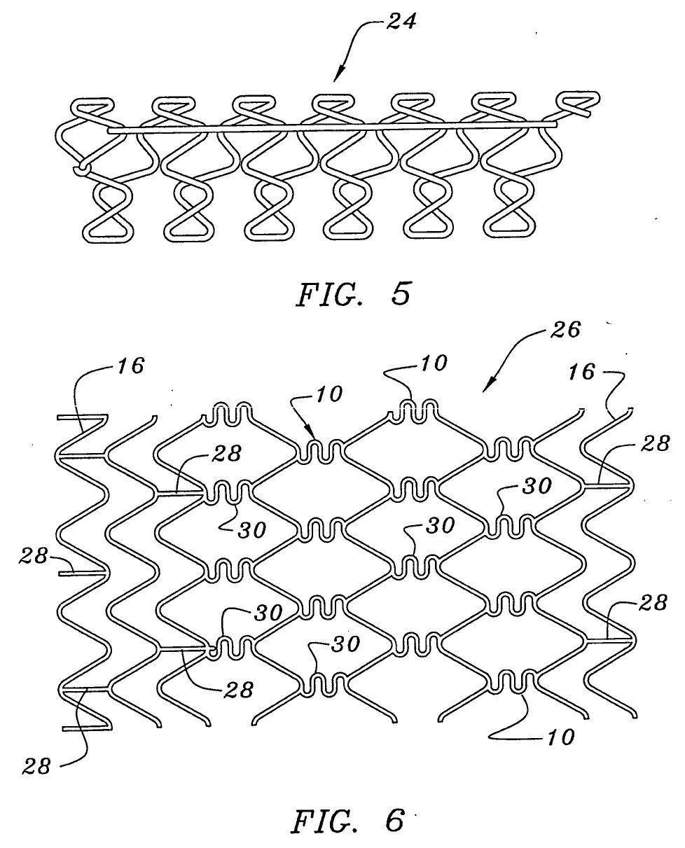

Referring to FIG. 4, a stent 16 includes a series of cells 18. The cells 18 are shown as circumferential sets of strut members forming an “open” cell stent 16. The circumferential sets of strut members are interconnected with connecting struts 28. Furthermore, at least on one section 20 of the open cell stent 16 can include a radiopaque coating 22 on at a portion of the cell 18. The radiopaque coating 22 can provide an increased visibility of the stent 16 by means of an x-ray, ultrasound, MRI, or other known viewing device.

Referring to FIG. 5, a coil stent 24 is provided. A coil stent 24 includes at least one curved segment which is arced about the longitudinal axis of the stent 24.

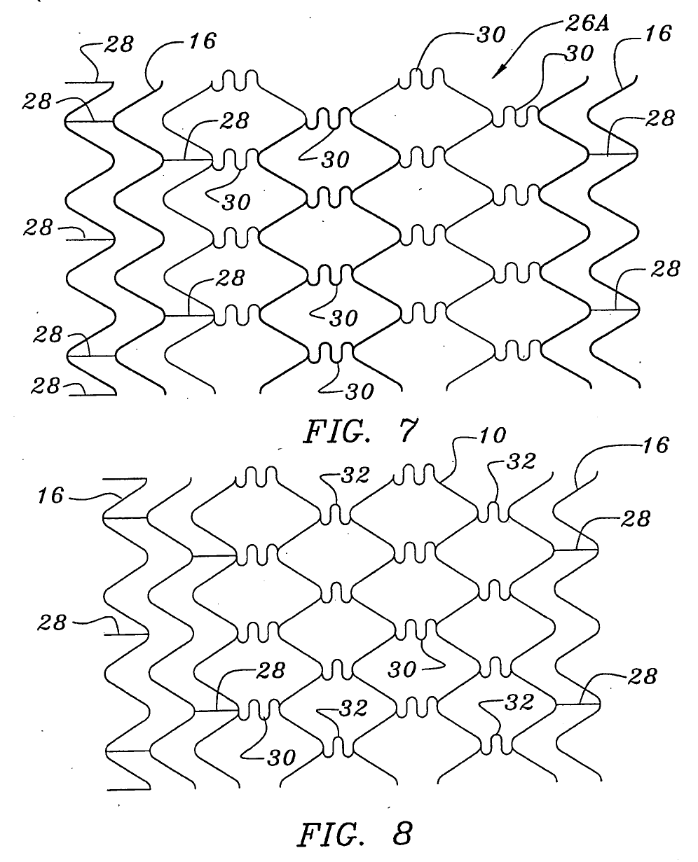

Referring to FIG. 6, a stent 26 is provided includes a plurality of interconnected cells of differentiating patterns. For example, first and second end portions of the stent 26 have a first pattern 16 and an intermediate portion of the stent 26 has a second pattern 10. The first pattern 16 can be in the form of an open cell configuration and the second pattern 10 be in the form of a closed cell configuration. Connecting struts 28 can join the patterns 10, 16 of the stent 26.

Referring to FIG. 7, a stent 26A is provided. The stent 26A includes a similar structure to stent 26, where the end portions of the stent 26 have an open cell configuration (first pattern) 16 and the intermediate portion of the stent 26 has a closed cell configuration (second pattern) 10. In the stent 26 of FIG. 6, the first and second patterns are depicted as having a uniform material thickness along the length of the stent 26. However, as shown in FIG. 7, the stent 26A can have a varying material thickness along the length of the stent 26A. For example, the first pattern 16 can have a greater material thickness than a material thickness of the second pattern 10. Similarly, the second pattern 10 can have a greater material thickness than the material thickness of the first pattern 16. Alternatively, the material thickness can vary within each of the patterns 10 and 16.

The closed cell configurations 10 further includes articulations 30, where the articulations 30 allow for expansion of the stent 26A. The articulation 30 can be provided in a variety of patterns. For example, the articulations 30 can be provided in a W-pattern. Additional articulation 30 patterns are disclosed in U.S. Pat. No. 6,375,677 to Penn et al, the contents of which are herein incorporated by reference in its entirety.

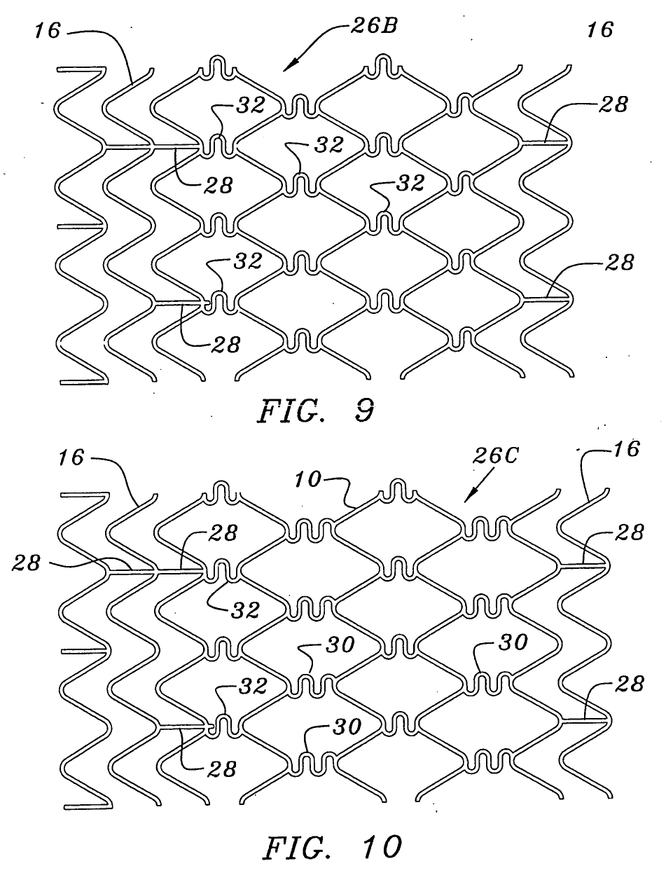

Referring to FIG. 8, the closed cells 10 can include a plurality of differing shaped articulation. For example, a number of the closed cells 10 can include articulations 30 having a first pattern, the W-pattern, and articulations 32 having a second pattern, an S-pattern.

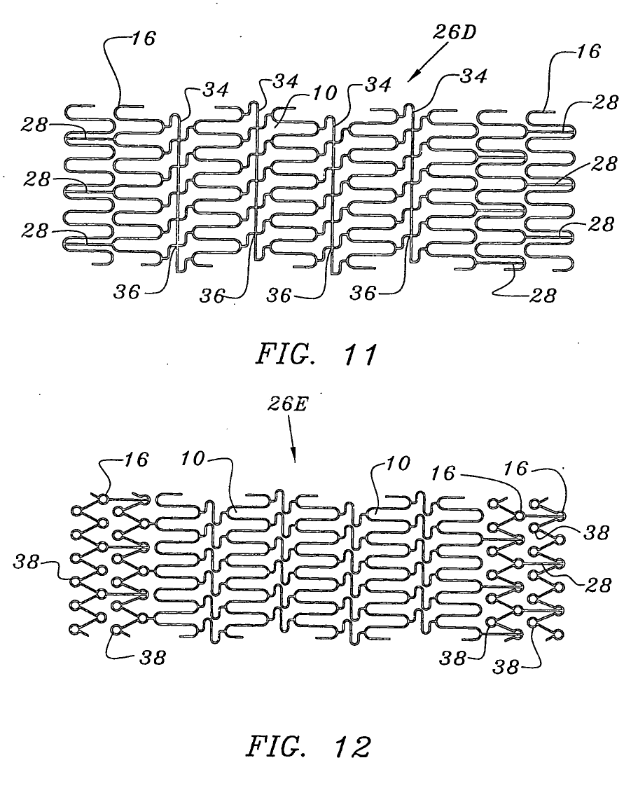

Further, non-limiting, exemplary cell and articulation patterns are as follows. In FIG. 9, the stent 26B has a closed cell design 10 at its mid-portion and an open cell design 16 at each end. The articulations 32 are all in the shape of an S-pattern. In FIG. 10, the stent 26C has a closed cell design 10 at its mid-portion and an open cell design 16 at each end, but with alternating S-pattern 32 and W-pattern 30 articulations. In FIG. 11, the stent 26D has an open cell design 16 at its ends in an S-pattern, a straight articulating member 34, a closed cell 10 mid-portion with a complex plus sign pattern articulation 36. In FIG. 12, the stent 26E has an open cell design 16 at its ends with a circle 38 in the open cell design. The center portion is a closed cell design 10.

Referring to FIG. 13, the stent 26F includes first and second patterns 16 and 10 having varying material thickness. The end portion of the stent 26F includes an open cell configuration. The open cell configuration 16 includes a portion having a thick 40 material thickness and another portion having a thin 42 material thickness. Similarly, the mid portion includes a closed cell configuration 10, which can include portions having a thick 40 material thickness and a thin 42 material thickness. For example, the articulations 32 of the closed cell configuration 10 can have a thick 42 material thickness.

The thickness of the open cell design 16 versus the closed cell design 10 may vary as seen in the drawings. For example, the open cell design 16 can be twenty-five percent thicker than the mid-portion or closed cell design 10.

The combination of an open cell 16 and closed cell 10 stent design creates a stent having both flexibility and radial strength along the length of the stent. The variable stent thickness 40 and 42 provides greater functional properties for coating the stent. If the coating is to enhance the radio opacity, then the ends can be made more radiopaque than the mid-portion. Furthermore, when the stent is coated with a pharmaceutical agent, the thick material can allow for an increased dosage of the pharmaceutical agent to be coated onto the stent. For example, as restenosis occurs in a stent invariably at its ends, a higher pharmaceutical concentration at the ends can more thoroughly inhibit such restenosis.

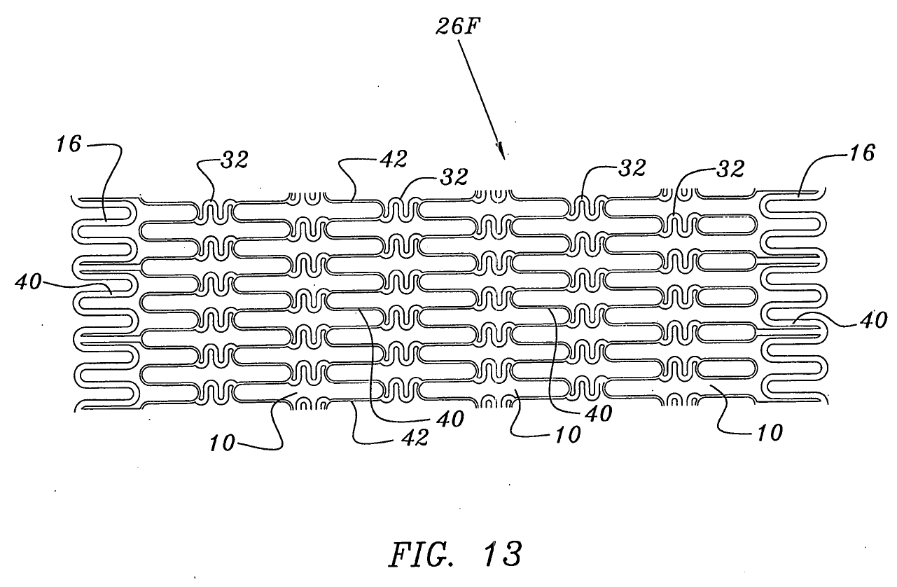

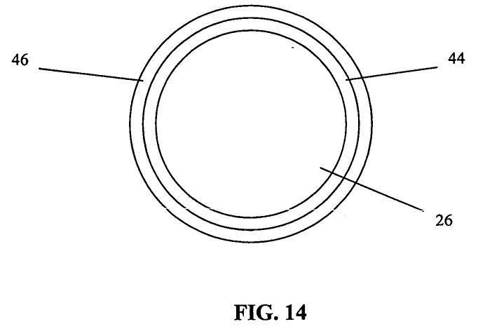

Referring to FIG. 14, the stent 26 can include a plurality of coatings. For example, the stent 26 can include two layers of coatings, a base coat 44 of metal and a top coat 46 of metal enhances radio opacity of the stent 26. Alternatively, the base coat 44 can be a polymeric or non-polymeric coating having a top coat 46 which can include a pharmaceutical agent. The pharmaceutical agent can slowly diffuse through the top coat 46 of the stent 26 over a period of time. The variable thickness design of the stents 26-26F can allow for a greater quantity of the pharmaceutical agent to be loaded onto the thick 42 sections of the stent 26, which can facilitate a graded release profile. For example, as noted above, the open cell 16 end portion of the stents 26-26F can have a thick 42 material thickness allowing for a greater quantity of the pharmaceutical agent to be coated onto the end portions of the stents 26-26F.

A coating of at least two layers over the base metal has a depth not to exceed ten microns. Typical coatings are set forth in U.S. Pat. Nos. 5,759,174; 5,725,572; 5,824,056; and 5,871,437 and are herein incorporated by reference.

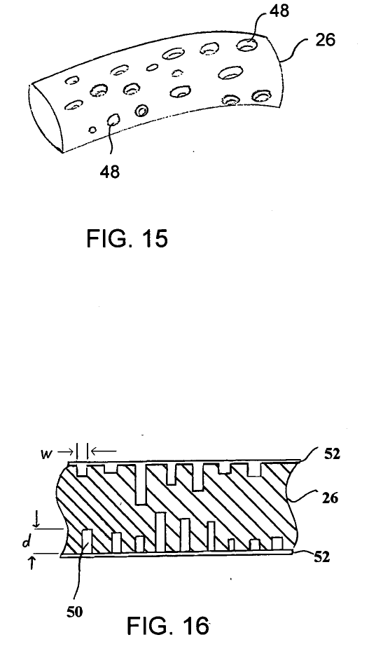

Referring to FIGS. 15 and 16, the stents 26-26F may include a plurality of reservoirs 48. The reservoirs 48 are dimensioned to receive a pharmaceutical agent 50 therein. The reservoirs 48 are sized to have a volume of at least 1 μg. A coating 52 can be provided to cover the reservoirs 48. The coating 52 can be absorbable or non-absorbable material with the pharmaceutical agent 50 released by diffusing through the coating 52. The coating 52 can be sufficiently permeable to selectively, controllably, release the pharmaceutical agent 50. Alternatively, for an absorbable coating 52, the pharmaceutical agent 50 is released as the coating 52 is absorbed. Alternatively, the coating 52 is coatings 44 and/or 46. The drug 50 is released by slowly diffusing through the coatings 44 and/or 46.

The reservoirs 48 have an opening with a diameter “w” and a depth “d.” The opening of each of the reservoirs 48 may have a uniform diameter “w,” or in the alternative, the opening of each of the reservoirs 48 may have non-uniform diameters “w.”

Similarly, each of the reservoirs 48 may have a uniform depth “d,” or in the alternative, the depth of the each of the reservoirs 48 may be non-uniform. The depth “d” of the reservoir is less than the thickness of the stent material, such that an individual reservoir 48 does not pass completely through the stent material. The reservoir 48 can be formed on the stent by laser cutting, chemical etching, or other related techniques.

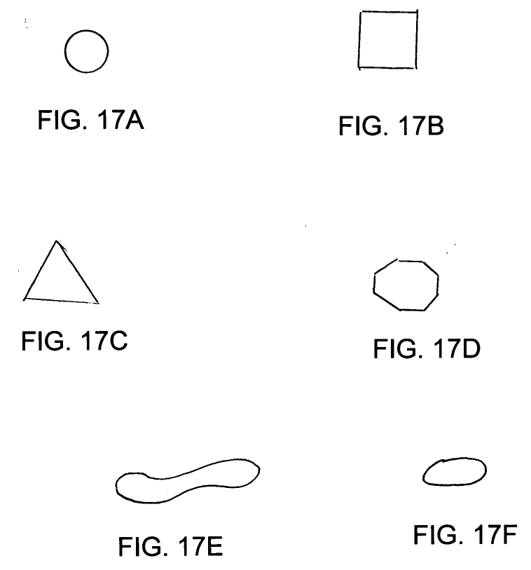

Referring to FIGS. 17A-17F the reservoirs 48 can have circular, elliptical, rectangular, triangular, polygon, or other geometric cross sectional area. Alternatively, the reservoirs 48 can have a free-formed cross-sectional area.

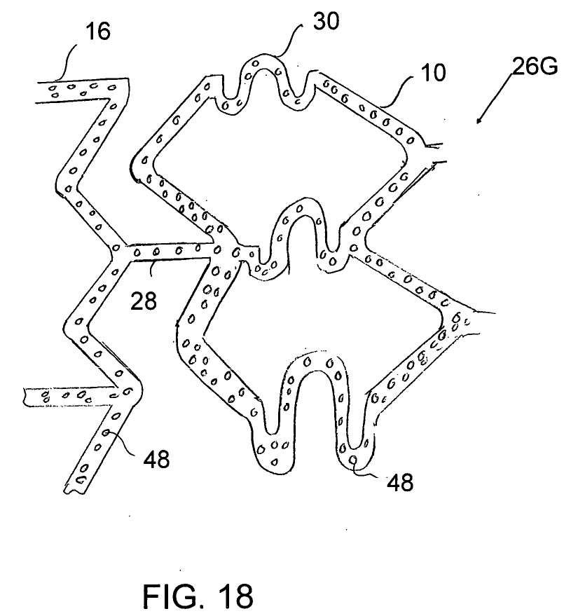

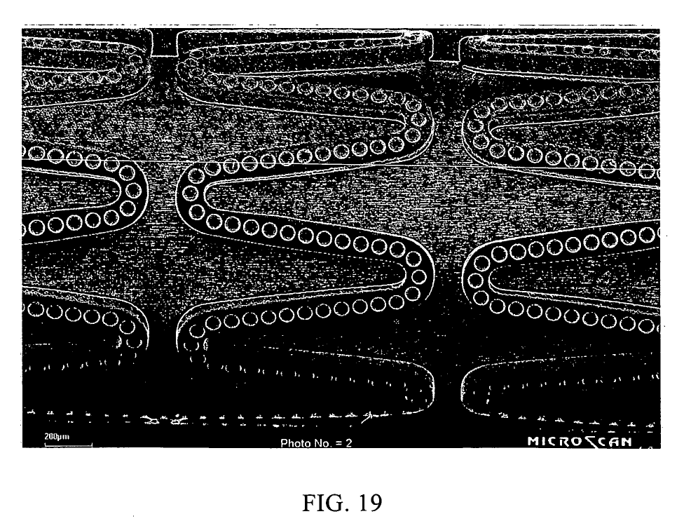

Referring to FIG. 18, the reservoirs 48 can be selectively positioned along the length of the stent 26G. For example, the reservoirs 48 can be positioned in the open cell 16 end portions, the closed cell 10 mid-portion, the articulations 30, the connecting struts, or any combinations thereof. Exemplary configurations include, positioning the reservoirs 48 only on the end portions 16, or only on the mid-portion 10. However, it is contemplated that other reservoir 48 configurations can be utilized.

Additionally, the selective positioning of the reservoirs 48 further includes controlling the size and density of the reservoirs on each of the stent 26G sections. For example, as restenosis occurs in a stent invariably at its ends, a higher pharmaceutical agent 50 concentration at the ends can more thoroughly inhibit such restenosis. The open cell 16 end portions can have greater reservoir 48 sizes than the closed cell 10 mid-portion of the stent 26G, allowing for a greater pharmaceutical agent 50 concentration to be provided at the end-portions 16 than at the mid-portion 10 of the stent 26G. Alternatively, the open cell 16 end portions can have greater reservoir 48 densities than the closed cell 10 mid-portion of the stent 26G, allowing for a greater pharmaceutical agent 50 concentration to be provided at the end-portions 16 than at the mid-portion 10 of the stent 26G.

It is further contemplated that the reservoirs 48 can be used in combination with the thick 42 and thin 40 materials sections of stent 26-26F. The thick 42 material sections of the stent can allow for increased reservoir 48 sizes and densities to be provided thereon, such that the thick 42 sections of the stent can have a greater pharmaceutical agent 50 concentration than on the thin 40 sections of the stent.

Similarly, the reservoirs 48 can be used in combination with the coating 44 and 46 of FIG. 14. As discussed above, the coatings 44 and 46 can be used to cover the reservoirs 48, wherein the pharmaceutical agent 50 is released by diffusing through the coating 44 and 46. The combination of the coating 44 and 46 and the selective positioning of the reservoirs 48 can be utilized to control the concentration of and release rate of the pharmaceutical agent 50.

As noted above, the coating 46 can similarly include a pharmaceutical agent 50. Where it is desired to have an increased pharmaceutical agent 50 concentration, reservoirs 48 can be provided to be used in combination with the coating 46.

The reservoirs 48 and the coating 46 can include the same pharmaceutical agent 50. Alternatively, the reservoirs 48 and the coating 46 can include different pharmaceutical agents, where the different pharmaceutical agent can be selectively positioned on the stents.

It is additionally contemplated that the reservoirs 48, coatings 44 and 46, and the thick 42 and thin 40 material thickness can be used individually or in combination to control the pharmaceutical agent 50 concentration along the stent.

The stents 26-26G of this invention are longitudinal, cylindrical, metal structures having at least an open cell and closed cell design joined together by struts. The metal can be nickel-titanium alloy (NITINOL) titanium, stainless steel or a noble base metal. In an exemplary embodiment, a NITINOL tube is laser-cut to form a structurally variable stent of the present invention.

In the embodiments previously described, the stent may or may not include a polymeric material to carry the therapeutic agent, to act as a base coat for the stent body, or to act as a top coat over the agent. In another exemplary embodiment, the stent includes no polymeric material. Polymers on stents may be the cause of allergic reactions experienced by stent recipients. The allergic reactions may include a rash, hives, itching, and even more seriously, difficulty breathing and fevers. Therefore, the stent of the present invention may be polymer-free.

Unlike prior art stents which include a polymeric material having a therapeutic agent dispersed or added to it, the agent(s) of the present invention are placed in reservoirs or are placed in the reservoirs and on the surface of the stent, without the use of polymeric material. The agent(s) may be added to a solvent such as Dulbecco's modified Eagle medium (DMEM), dimethyl sulfoxide (DMSO), and/or ethyl alcohol (EtOH). The agent-solvent mixture may be disposed on the stent body and within the reservoirs. The solvent dissipates or evaporates leaving the agent(s) on the stent. Alternatively, or additionally, the agent may be placed on the stent and within a reservoir with a biocompatible adhesive. The adhesive may be biostable or bioerodible. With or without an adhesive holding the agent on the stent, a top coat may be placed over the stent to protect the agent(s) from handling during surgery. The top coat may also control the release rate of the agent(s) from the stent. The top coat may be biostable or bioerodible.

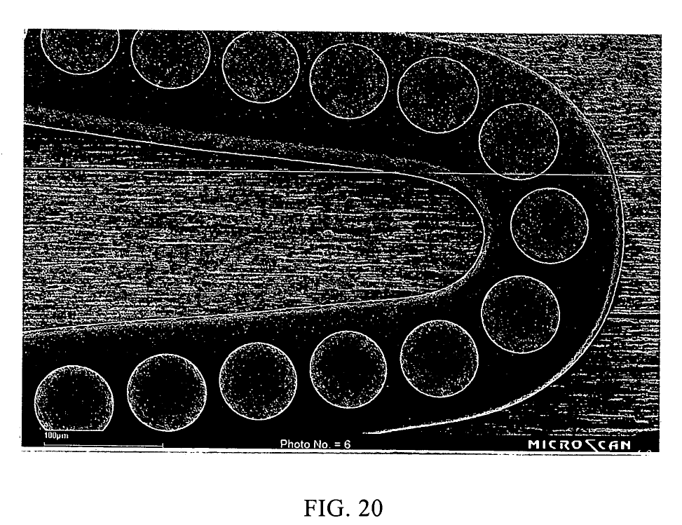

FIG. 19 is a photograph of a portion of stent. The stent has an open-cell pattern. A plurality of reservoirs is disposed on the exterior surface of the stent. Each reservoir has a concave, partially spherical design or an inverted dome shape. FIG. 20 is a close-up photograph of the reservoirs of the stent of FIG. 19. The reservoirs or bucket shaped cavities receive one or more therapeutic agents for delivery of the agents to a vessel wall when the stent is implant. It is contemplated that the reservoirs may be placed on any surface of the stent. Alternatively, the reservoirs may be uniformly placed only in the main portion of the stent and not on the bridges.

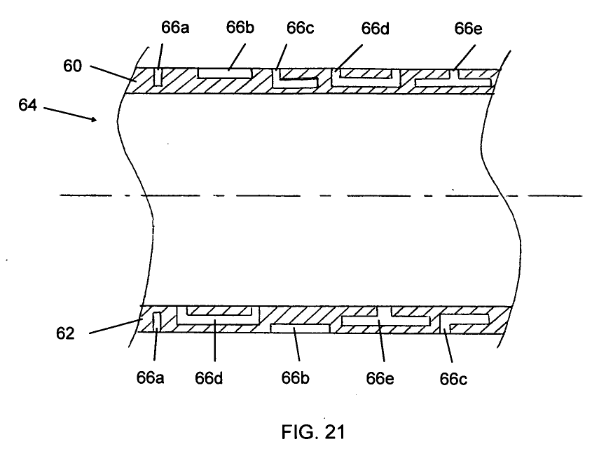

Referring now to FIG. 21, walls 60, 62 of a stent 64 includes reservoirs and tunnels 66 dimensioned and configured to hold and release a therapeutic agent. The walls shown in the FIG. 21 may be from any stent design, such as a structurally variable stent or a stent design previously disclosed herein. Furthermore, the walls 60, 62 may represent any portion of a stent. That is, the cross-sectional view may be that of a tubular stent body, a portion of a pattern design, a connecting member, a strut, a circumferential band, circumferential sets of struts/bands, an end portion, or a mid-portion. The walls include various configurations of reservoirs and tunnels. Reservoir 66a extends generally perpendicular to the exterior wall surface. Reservoir 66b is a groove extending generally parallel to the wall. Reservoir 66c is L-shaped. Reservoir 66d is U-shaped. Reservoir 66e is T-shaped. Each reservoir design provides a unique therapeutic agent release profile. Comparing reservoirs 66a and 66b, the agent in 66a will be released slowly but over a longer time period, while the agent in 66b will be released rapidly for a shorter duration. The agent in 66c will release at a similar rate as the agent in 66a and will release for a duration similar to the agent in 66b. The agent in 66d is released from two openings. Therefore, the agent will release about twice as fast as the agent in 66a and will last about as long as the agent in 66c. The design of 66e provides a slow release like 66a and provides the longest duration of release time.

The reservoirs 66 of the lower wall 62 of FIG. 21 have similar configurations. In the lower wall, reservoirs 66d and 66e have openings that permit the agent(s) therein to release inward into the vessel or blood stream. Reservoirs 66a, 66b, and 66c open away from the stent 64 to release agent(s) to the vessel wall. It is contemplated that the reservoirs and tunnels of FIG. 21 may be formed within a wall of a stent using lasers and other suitable technology known to those with ordinary skill in the art.

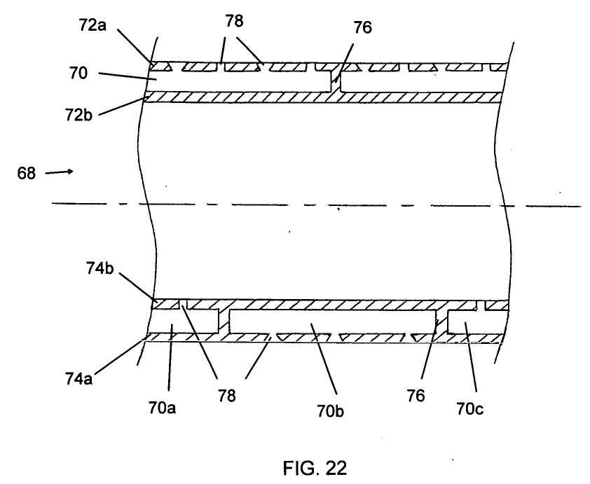

In FIG. 22, an embodiment of a double-walled stent 68 having a reservoir(s) 70 is illustrated. The walls 72a, 72b and 74a, 74b shown in FIG. 22 may be from any stent design, such a structurally variable stent or a stent design previously disclosed herein. Furthermore, the walls may represent any portion of a stent. That is, the cross-sectional view may be that of a tubular stent body, a portion of a pattern design, a connecting member, a strut, a circumferential band, an end portion, or a mid-portion. The double-walled construction of the stent 68 of FIG. 22 creates a space or reservoir 70 between the walls. A support member 76 may be placed within the reservoir 70 between the walls to provide strength to the stent. Also, the double-walled configuration may or may not extend over the entire longitudinal length of the stent. Only a portion of the stent may be double-walled. For example, double walls may be at a mid-portion, at an end portion, at one circumferential band, at staggered circumferential bands, at struts, and/or at staggered struts.

As shown in FIG. 22, the upper portion of the stent 68 includes two walls 72a, 72b with a reservoir 70 therebetween. One or more therapeutic agents may be placed in the reservoir. A support column 76 may optionally be positioned between the walls. The support column 76 may be configured to divide the reservoir 70 into two distinct reservoirs or may be configured to allow open communication throughout the reservoir. Shown in the upper portion of the stent 68, the external wall 72a includes openings 78 which allow an agent(s) from the reservoir 70 to be dispersed to the vessel wall when the stent is implanted. The openings 78 may be straight channels or may be flared channels. Flared channels permit the agent to be released to a large area of the vessel wall. As seen in the lower portion of the stent 68, both the external and internal walls 74a and 74b include openings 78. In this configuration, the support columns may divide the reservoir into discrete areas. Areas 70a and 70b of the reservoir may include an agent(s) that may be released into the fluid stream. Area 70c of the reservoir may include an agent(s) that may be dispersed to the vessel wall.

It is contemplated that an agent(s) in a reservoir may be released both into the fluid stream and into the vessel wall. Such a reservoir would have openings in both the external and internal walls. It is also contemplated that a reservoir may have a longitudinal partition. Therefore, one or more agents may be placed in the reservoir between the partition and the inner wall to allow dispersion of the agent(s) into the fluid stream, while one or more agents may also be placed in the reservoir between the partition and the outer wall to allow release of the agent(s) into the vessel wall. The double-wall construction of the stent may be formed by using lasers, by connecting two stent walls generally parallel to each other and spaced apart from each other, or by using other suitable technology known to those with ordinary skill in the art.

Stent Preform

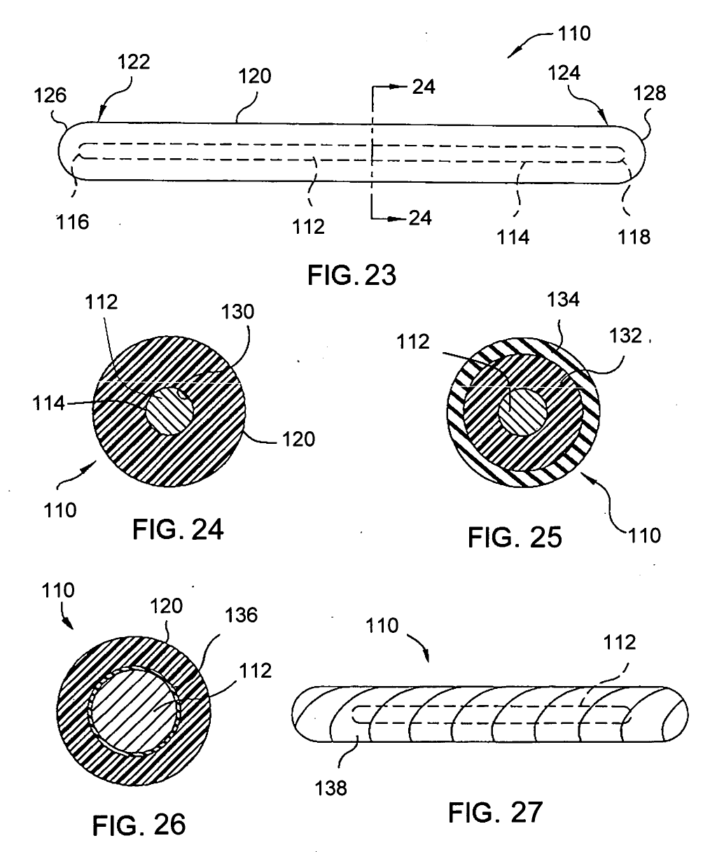

As seen in FIG. 23, the present invention also provides for a stent preform 110 which takes the form of a wire or core 112 with a contact surface 114 and core ends 116 and 118. The core 112 of the stent preform 110 is preferably made of a rigid or rigidizable material. It may additionally be formed of a material that exhibits suitable ductility, with the material further being chosen based on its radiopacity in order to allow x-ray imaging. Various metals are appropriate for the substrate core, including but not limited to stainless steel, titanium, nickel, and combinations and alloys thereof. In particular, alloys that display the “shape memory” effect, such as a Ni 50% Ti alloy and several copper-base alloys, are appropriate. In an exemplary embodiment, NITINOL is used for the core 112. As known to those skilled in the art, proper heat treatment of shape memory alloys allows structures to be created which assume several configurations depending on the temperature. Thus, a first shape can be used to facilitate implantation of the stent, and warming of the stent in the body lumen permits the stent to transform to a second shape that provides mechanical support to an artery. The second shape may be in the form of a coil to embolize a part of the anatomy or close a duct, or a mechanical scaffolding structure for vascular or nonvascular purposes. Also, cobalt-based alloys such as Eligiloy may be used as a metal core.

Other stiff materials can also be used to form the core 112, including carbon fibers, Kevlar, glass fibers, or the like. Some fiber filaments may not retain enough memory to maintain a preselected stent or coil shape. Thus, the stent may be fabricated by braiding several such filaments together to form a tubular structure. The filaments may be stretched to create a low profile, while releasing the filament from a stretched state allows it to assume a desirable shape. As is known to those skilled in the art, various braiding techniques may be employed, as well as various polymers or fillers. The core 112 is preferably substantially cylindrical in shape, although other core cross-sections may be used such as rectangular or hexagonal configurations.

As further seen in FIG. 23, the core 112 is surrounded by an outer sheath 120 having sheath ends 122 and 124 and caps 126 and 128. The sheath 120 includes a therapeutically effective amount of an agent or agents to treat a disease process in the pathology of a vascular disease. The agent or agents may include a macrolide immunosuppressant, anti-inflammatory agent, non-proliferative agent, anti-coagulant, anti-platelet agent, Tyrosine Kinase inhibitor, anti-infective agent, anti-tumor agent, anti-leukemic agent and a combination thereof. Examples of such agents are subsequently provided. The sheath 120 may also serve as a sleeve or jacket which surrounds the core 112 to prevent the core from directly contacting a wall of a body lumen. The sheath 120 is preferably thin, and preferably an ultrathin tube of extruded polymer which may be microporous or macroporous. Although the sheath 120 may even have a thickness on the submicron level, in a preferred embodiment the sheath 120 has a thickness of between about 0.1 microns and 5 millimeters. The outer sheath 120 may be heat treated to ensure adhesion or bonding of the sheath 120 to the core 112. It may also be necessary to heat the composite to melt the polymer and permit it to flow, thereby not only allowing more effective bonding with the core 112 but also filling any gaps that may exist that expose the core 112.

Suitable polymers for the stent preform include biocompatible polymers of particular permeability. The polymers can form a permeable, semi-permeable, or non-permeable membrane, and this property of the polymer may be selected during or after extrusion depending upon the particular polymer chosen. As shown in FIG. 24, the sheath 120 has an interior surface 130, which closely communicates with the contact surface 114 of the core 112. Numerous polymers are appropriate for use in stent preform 110, including but not limited to the polymers PTFE, ePTFE, PET, polyamide, PVC, PU, Nylon, hydrogels, silicone, silk, knitted or woven polyester fabric, or other thermosets or thermoplastics. In a preferred embodiment, the polymer is selected as a heat-shrinkable polymer. The sheath 120 may also be in the form of a thin film, which is deposited over the entire surface of core 112. A layer or multiple layers of submicron particles (nanoparticles) may also create a nanotube surrounding core 112. The sheath 120 must completely encapsulate core 112, and thus areas of the sheath form caps 126 and 128, as seen in FIG. 23.

The outer sheath 120 may be knitted or woven to form a braided configuration, however a sheath formed in this manner must still completely encapsulate the core 112. Sufficient tightness of the braiding around the core 112 is required, or alternatively the strands may be sealed together to form a continuous surface after braiding. The braided configuration is also designed to cover the ends 116 and 118 of core 112, as seen in FIG. 23.

FIG. 25 shows the outer sheath 120 formed of several layers of material. The layers may be of the same or varying thickness, and may be the same or different materials. In an exemplary embodiment, a layer 132 is formed of a first polymer, and another layer 134 is a biological or other synthetic veneer which can preserve blood function. However, the biological material must be able to completely encapsulate the core 112, even after the core has been coiled or braided and formed into the shape of a stent. Thus, the biological coating should resist tearing and delamination which could result in exposure of core 112. If such a coating is applied prior to shaping the preform into a stent, it should be capable of withstanding the deformations and stresses that are induced by coil winding or braiding machines. It should also be capable of withstanding elevated temperatures if heat treatments are necessary.

The veneer may be an anticoagulent material such as heparin, coumadin, tichlopidiene, and chlopidogrel. The veneer may also be a genetic material such as angiogenic factors, tissue inhibiting material, growth factors such as VEGF, PDGF, and PGF, as well as thrombin inhibiting factors. The growth factors and angiogenic factors can be sourced biologically, for example through porcine, bovine, or recombinant means, and the growth factors even can be derived from the patient's own body by processing blood from the patient. The veneer may be applied to the polymer layer by dipping the outer sheath 120 into growth factors for several minutes to promote attachment, and additional factors may be added to help effectuate the attachment. The growth factors can further be encapsulated in a release mechanism made of liposomes, PLA, PGA, HA, or other release polymers. Alternatively, the growth factors can be encapsulated in non-controlled release, naturally-derived polymers such as chitosan and alginate.

In an alternate embodiment, the veneer can be sandwiched between the micropores of the polymer layer so that a controlled release occurs. In yet another alternate embodiment, a multilayer outer sheath 120 can be formed wherein an active release substrate polymer is attached to a layer of a different polymer, or sandwiched between two layers of either the same or different polymers. The outer sheath 120 may otherwise be formed of an inert polymer, or of an inert polymer surrounding an active polymer.

FIG. 26 shows another embodiment of the stent preform 110 according to the present invention. The stent preform 110 includes a core 112, an outer sheath 120, and a lubricious lining 136. The lubricious lining 136 is disposed between core 112 and outer sheath 120 to facilitate insertion of core 112 into the sheath 120. The lubricious lining 136 may be attached to core 112 or outer sheath 120, or it may be separate. The lining 136 permits a tight fit between core 112 and outer sheath 120 by providing a lubricated surface on which either can be slid relative to the other, thereby allowing the inner dimension of the outer sheath 120 to very closely match the outer dimension of the core 112.

In addition to applying a therapeutic coating or sheath to an intravascular implant, the implant can include therapeutic tape where the tape includes a therapeutic agent or agents to treat or prevent a disease process in the pathology of a vascular disease. The agent or agents may include a macrolide immunosuppressant, anti-inflammatory agent, non-proliferative agent, anti-coagulant, anti-platelet agent, Tyrosine Kinase inhibitor, anti-infective agent, anti-tumor agent, anti-leukemic agent and a combination thereof. The intravascular implant may be, but is not limited to, a balloon catheter, stent, stent graft, stent preform, drug delivery catheter, atherectomy device, filter, scaffolding device, anastomotic clip, anastomotic bridge, suture material, metallic or non-metallic wire, embolic coil or a combination thereof.

For example, FIG. 27 shows a stent preform 110 with the core 112 wrapped in tape 138. The tape 138 completely covers core 112 so that the core is isolated from the lumen walls after implantation. In an alternate embodiment, the tape 138 is applied around a core that is already covered with another coating or layer of polymer. The tape 138 may be applied to the core using a winding machine or other suitable means.

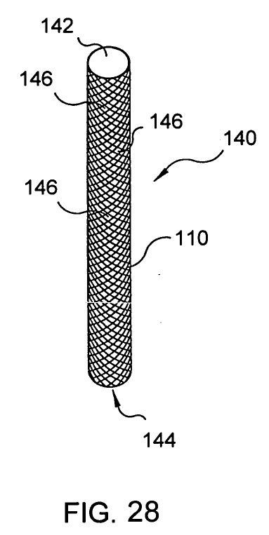

FIG. 28 shows a braided stent 140 made from a stent preform 110. In an alternative embodiment of braided stent 140, multiple stent preforms 110 may be used. The ends 142 and 144 may be pulled to extend the braided stent 140 to a longer length, thereby also decreasing the inner diameter of the stent. When released, the stent returns to a relaxed length and diameter. Open areas 146 between the stent preform walls permit new tissue growth which may eventually cover the stent structure. The braided stent, or other shapes or coils forming a stent, can be mounted on top of an expansile device such as a balloon catheter, which expands the stent from a relaxed diameter to an elongated diameter. A stent 140 formed from at least one stent preform 110 can prevent or treat a disease process or processes in the pathology of a vascular disease. The therapeutic agent or agents of the stent preform 110 may include a macrolide immunosuppressant, anti-inflammatory agent, non-proliferative agent, anti-coagulant, anti-platelet agent, Tyrosine Kinase inhibitor, anti-infective agent, anti-tumor agent, anti-leukemic agent and a combination thereof.

A delivery housing in combination with a shaft may be used to insert the stent into a lumen. The housing may have a cylindrical shape, and the stent, loaded on the shaft in extended state, is placed in the housing. Once the housing is inserted into the lumen, the stent may be slowly withdrawn from the housing while supported and guided by the shaft, and allowed to return to its unextended shape having a greater diameter. The housing and shaft are completely withdrawn from the lumen leaving the stent as a lining inside the vessel wall to exclude blockage from the vessel. By emobilizing the duct with a stent having an isolated core, the stent is more readily accepted by the body. This implantation method can be applied to any anatomical conduit.

Stents incorporating shape memory materials may be heat treated in various states to permit the stretched configuration. Although the core may require treatment at 650 degrees Celsius, care must be exercised when fabricating the stents of the present invention since a polymer overlayer will be provided.

Stent preforms may be spooled to permit storage in a roll form, or may also be kept in an unrolled state.

U.S. Pat. No. 6,475,235 issued on Nov. 5, 2002 and entitled, “Encapsulated Stent Preform” further discusses an outer sheath and tape for covering an implant, and more particularly, discusses a stent preform. Also, U.S. Pat. No. 6,746,478 issued on Jun. 8, 2004 discloses a stent formed from encapsulated stent preforms. The disclosures of those patent documents are incorporated herein by reference.

The stent preform previously described includes a sheath which may have a polymeric material. As noted above, polymers may be the cause of allergic reactions in patients receiving implants having polymeric material. These allergic reactions can be severe and, in some case, can lead to death. Therefore, in another exemplary embodiment, a stent preform is provided which is free from polymeric material.

Unlike a stent preform having one or more therapeutic agent dispersed in or added to a polymer, the therapeutic agents of a polymer-free stent preform is placed in reservoirs. The agent(s) may be added to a solvent such as DMEM, DMSO, and/or EtOH. The agent-solvent mixture may be disposed on the stent preform and within the reservoirs. The solvent dissipates or evaporates leaving the agent(s) on the stent preform. Alternatively, or additionally, the agent may be placed on the stent preform and within a reservoir with a biocompatible adhesive. The adhesive may be biostable or bioerodible. With or without an adhesive holding the agent on the stent preform, a top coat may be placed over the preform to protect the agent(s) from handling during surgery. The top coat may also control the release rate of the agent(s) from the stent preform. The top coat may be biostable or bioerodible.

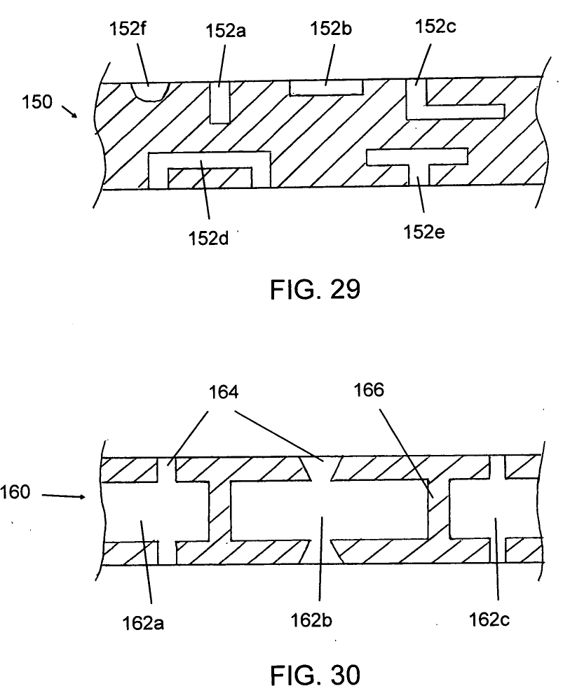

Referring now to FIG. 29, a polymer-free stent preform 150 includes reservoirs and tunnels 152 dimensioned and configured to hold and release a therapeutic agent. As previously described, the preform takes the form of a filament or wire. The preform 150 includes various configurations of reservoirs and tunnels. Reservoir 152a extends generally perpendicular to the exterior wire surface. Reservoir 152b is a groove extending generally parallel to the wire. Reservoir 152c is L-shaped. Reservoir 152d is U-shaped. Reservoir 152e is T-shaped. Reservoir 152f is concave shaped. Each reservoir design provides a unique therapeutic agent release profile.

The plurality of reservoirs of the stent preform may be aligned circumferentially about the wire, longitudinally along the wire, and/or randomly placed about the surface of the wire. It is contemplated that the reservoirs, tunnels, and openings of FIG. 29 may be formed within a stent preform using lasers and other suitable technology known to those with ordinary skill in the art.

In FIG. 30, an embodiment of a hollow stent preform 160 is illustrated. The hollow center of the wire functions as the therapeutic agent reservoir 162. Openings or passageways 164 extend from the reservoir and through the outer wall of the wire to allow the agent within the reservoir to exit the stent preform. The openings 164 may be straight channels or may be flared channels. Flared channels permit the agent to be released to a large area of the vessel wall. Support members 166 may be placed in the reservoir to give the preform structural support. The support members 166 may be elongated rods, or similar shape, thereby creating a reservoir with open communication throughout. Alternatively, or in addition, the support member 166 may be disc shaped. In this configuration, the disc support positioned within the reservoir creates a wall dividing the reservoir into multiple discrete areas. Areas 162a, 162b, and 162c of the reservoir may include the same or different therapeutic agents.

Also, the hollow configuration of the stent preform may or may not extend over the entire longitudinal length of the wire. Only a portion of the preform may be hollow. For example, the preform may be hollow at a mid-portion or at an end portion of the wire. Also, the preform may be hollow at one or more portions and may include reservoirs like those of FIG. 29 at one or more other portions. It is contemplated that the reservoirs (hollow area) and openings of FIG. 30 may be formed within a stent preform using lasers and other suitable technology known to those with ordinary skill in the art.

Implant Coatings

In a related invention, a coating for an intravascular implant, such as a structurally variable stent or a stent preform, is provided. The coating can be applied either alone, or within a polymeric matrix, which can be biostable or bioabsorbable, to the surface of an intravascular device. If a polymeric matrix is applied, such an implant may be selectively used in patients who do not obtain allergic reactions to polymeric material. The coating can be applied directly to the implant or on top of a polymeric substrate, i.e. a primer. If desired, a top coat can be applied to the therapeutic coating.

The therapeutic intravascular implant coating may include an effective amount of at least one therapeutic agent to treat or prevent a disease process of a vascular disease of a patient, wherein the effective amount of at least one therapeutic agent cures the vascular disease.

Alternatively, the intravascular implant coating may include a therapeutically effective amount of a first agent, the first agent acting on a calcium independent cellular pathway, and a therapeutically effective amount of a second agent, the second agent acting on a calcium dependent cellular pathway. The combined amount of the first and second agents treats or prevents hyperproliferative vascular disease. In an exemplary embodiment, the first agent may be a macrolide immunosuppressant, such as rapamycin, and the second agent may be cyclosporine A.

Instead of the second agent being cyclosporine A, the second agent may be an anti-inflammatory agent, non-proliferative agent, anti-coagulant, anti-platelet agent, Tyrosine Kinase inhibitor, anti-infective agent, anti-tumor agent, anti-leukemic agent, or a combination thereof. Examples of such agents are subsequently provided.

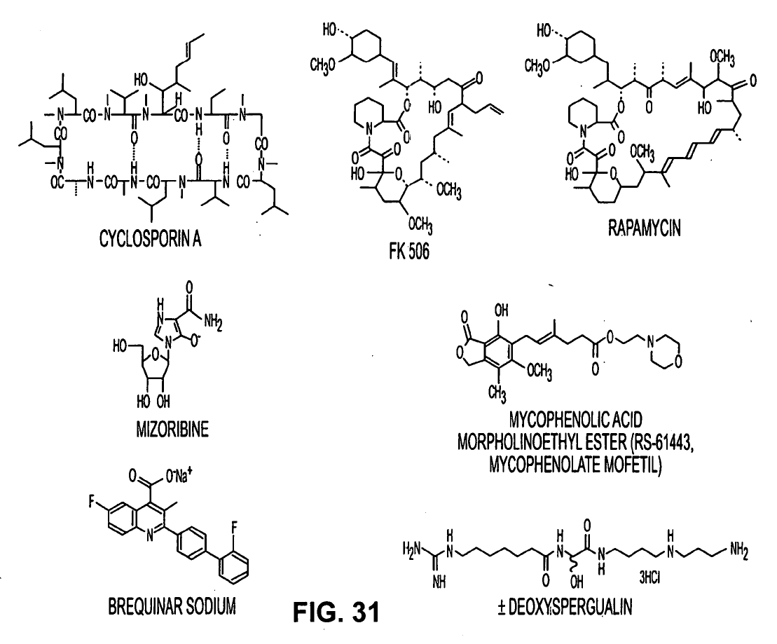

FIG. 31 shows some therapeutic agents and chemical structures used in the present invention. The distinct sites of action of rapamycin, which is a macrolide immunosuppressant acting on a calcium independent pathway, and cyclosporine A, which is an IL-2 transcription inhibitor acting on a calcium dependent pathway, and their relatively non-overlapping toxicities will enable this combination to be used intravascularly after angioplasty to prevent cellular growth at the site of injury inside the vessel.

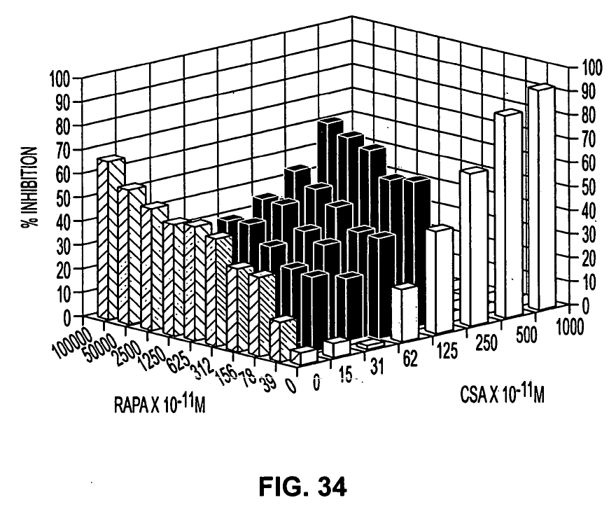

The rationale for a combinatorial therapy for intravascular therapy is at least in part as follows. The immunosuppressive efficacy to prevent allograft rejection after staggered administration of the two agents was similar to that obtained with simultaneous administration of combined therapy and significantly reduced the incidence of rejection in cardiac allografts (FIG. 34).

In the past, clinicians have learned to take advantage of known interactions between cyclosporine A and other compounds such as “azole” antifungals to reduce cyclosporine A dose requirements. In particular, the azole antifungals have no known clinically significant immunosuppressive properties and have little toxicity at the doses used in this context. Because in this context, they are not given for their pharmocodynamic effects. The amount of absorption of the azole antifungals is not critical. In the case of co-administration of cyclosporine A and rapamycin, both agents have low and variable bioavailabilities as well as narrow therapeutic indices. In addition, this interaction is dose dependent and can be completely avoided with low doses of combinatorial delivery.

In some aspects, the process of allograft rejection is similar to the restenosis process inside the coronary arteries after injury to the vessel wall. After arterial injury, multiple mitogenic and proliferative factors have been identified as capable of triggering signaling mechanisms leading to SMC activation. Because rapamycin and cyclosporine combination targets fundamental regulators of cell growth, it may significantly reduce restenosis.

A coating for an intravascular implant that includes the combination of rapamycin and cyclosporine A helps ensure that the mediation of cell growth happens very early in the cell cycle. For example, cyclosporine A acts early after T cell activation, thereby blocking transcriptional activation of early T cell specific genes. Rapamycin acts later in the cell cycle by blocking growth factor driven cell proliferation. The two agents can be provided in the coating such that the amount of rapamycin is higher than cyclosporine A. Thus, the ratio of rapamycin to cyclosporine A could be about 51% and above.

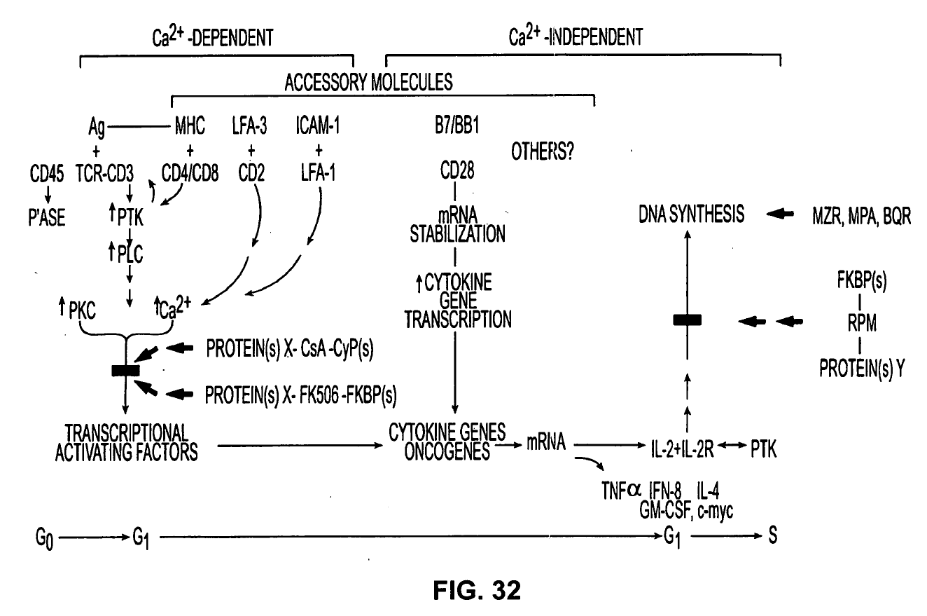

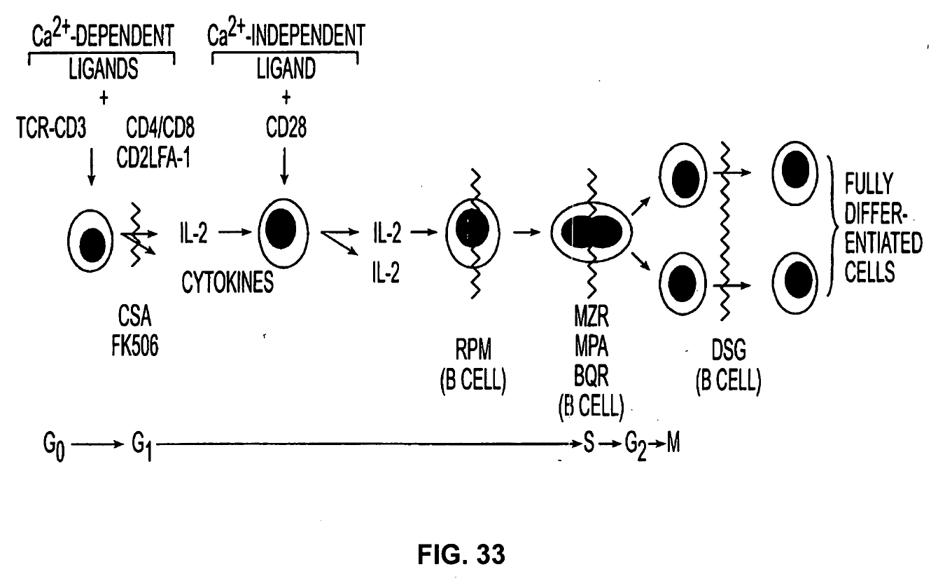

As shown in FIGS. 32 and 33, the activation of T cells, which seems to be critical for induction of host resistance and consequent rejection of the transplanted organ, occurs in three phases. The first phase causes transcriptional activation of immediate and early genes (IL-2 receptor) that allow T cells to progress from a quiescent (G0) to a competent (G1) state. In the second phase, T cells transduce the signal triggered by stimulating cytokines in both an autocrine and a paracrine fashion permitting entry into the cell cycle with resultant clonal expansion and acquisition of effector functions in the third phase of the immune response. Cyclosporine A inhibits the first phase and rapamycin inhibits the second phase of T cell activation. By ensuring that the stent surface or any intravascular surface has both these drugs, it is ensured that the restenotic response from the arterial wall is significantly reduced or is completely eliminated.

Although the two agents could be used separately, a considerable over dosing has to be done to ensure that both the agents have a necessary therapeutic effect. This overdosing could potentially result in side effects, which include improper healing of the vessel and also an incomplete intimal formation.

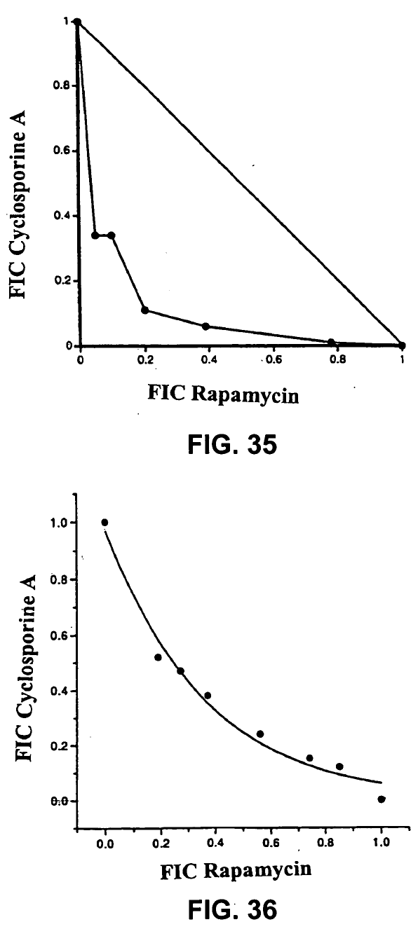

The combination of the agents would mean that both agents can be combined at a very low dosage and the combination would actually increase the therapeutic levels rather than administering monotherapy. This is illustrated in FIGS. 35 and 36, which shows the synergistic effects of rapamycin and cyclosporine A. The toxicity of the combination of agents is significantly reduced when both are combined together. Providing two agents that are active on two different cell cycles to prevent proliferation increases the therapeutic window of the agent. The combination actually increases the level of immunosuppression when compared to monotherapy.

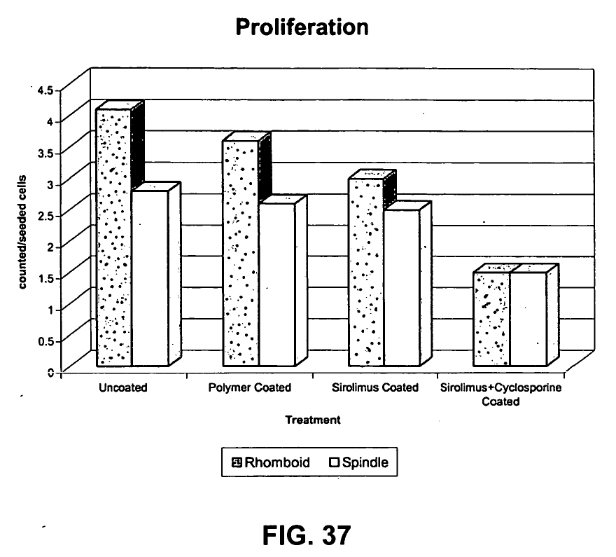

FIG. 37 illustrates the amount of proliferation of vascular smooth muscle cells based on different treatments. The vascular wall primarily consists of smooth muscle cells. The proliferation of these smooth muscle cells cause hyperproliferative vascular disease or restenosis. There are generally two types of smooth muscle cells: rhomboid shaped and spindle shaped. Rhomboid shaped cells are seen in a normal vessel wall, while spindle shaped cells are seen during restenosis (after balloon angioplasty or stenting). The graph of FIG. 37 shows that combinatorial therapy is more effective in preventing both types of smooth muscle cells than monotherapy. The graph shows an amount of proliferation which is less with rapamycin and cyclosporine (combinatorial therapy) than with rapamycin alone (monotherapy). Uncoated and polymer coated implants show greater amounts of proliferation when compared to combinatorial therapy.

Combinatorial therapy for delivery of more than one agent through a coating may be used on any intravascular implant. As used herein, implant means any type of medical or surgical implement, whether temporary or permanent. Delivery can be either during or after an interventional procedure. The intravascular implant may be, but is not limited to, a balloon catheter, stent, stent graft, stent preform, drug delivery catheter, atherectomy device, filter, scaffolding device, anastomotic clip, anastomotic bridge, suture material, metallic or non-metallic wire, embolic coil or a combination thereof. Non-limiting examples of coated, intravascular implants now follow.

The outside surface of a balloon catheter may be coated with the combination according to the present invention and could be released immediately or in a time dependent fashion. When the balloon expands and the wall of the vessel is in contact with the balloon, the release of the combination can begin. Small nanospheres of the agents can actually be transported into the vessel wall using the balloon so that these nanospheres ensure delivery over longer period of time.

The surface of a stent may be coated with the combination of agents and the stent is implanted inside the body. The stent struts could be loaded with several layers of the agents or with just a single layer. A transporter or a vehicle to load the agents on to the surface can also be applied to the stent. The graft material of the stent graft can also be coated (in addition to the stent or as an alternative) so that the material is transported intravascularly at the site of the location or the injury.

The drug delivery catheters that are used to inject drugs and other agents intravascularly can also be used to deliver the combination of agents. Other intravascular devices through which the transport can happen include atherectomy devices, filters, scaffolding devices, anastomotic clips, anastomotic bridges, suture materials etc.

The coating may be applied directly to the intravascular implant. Alternatively, the coating can be applied to a primer, i.e. a layer or film of material upon which another coating is applied. Furthermore, the first and second agents can be incorporated in a polymer matrix. Polymeric matrices (bioabsorbable and biostable) can be used for delivery of the therapeutic agents. In some situations, when the agents are loaded on to the implant, there is a risk of quick erosion of the therapeutic agents either during the expansion process or during the phase during with the blood flow is at high shear rates at the time of implantation. In order to ensure that the therapeutic window of the agents is prolonged over extended periods of time, polymer matrices can be used. Again, implants with polymeric material may be selectively utilized with patients not prone to polymer allergic reactions.

These polymers could be any one of the following: semitelechelic polymers for drug delivery, thermo responsive polymeric micelles for targeted drug delivery, pH or temperature sensitive polymers for drug delivery, peptide and protein based drug delivery, water insoluble drug complex drug delivery matrices, polychelating amphiphilic polymers for drug delivery, bioconjugation of biodegradable poly lactic/glycolic acid for delivery, elastin mimetic protein networks for delivery, generically engineered protein domains for drug delivery, superporous hydrogel composites for drug delivery, interpenetrating polymeric networks for drug delivery, hyaluronic acid based delivery of drugs, photocrosslinked polyanhydrides with controlled hydrolytic delivery, cytokineinducing macromolecular glycolipids based delivery, cationic polysaccharides for topical delivery, n-halamine polymer coatings for drug delivery, dextran based coatings for drug delivery, fluorescent molecules for drug delivery, self-etching polymerization initiating primes for drug delivery, and bioactive composites based drug delivery.

Regardless of whether the coating includes a polymer matrix and where it is applied (directly on the implant, on top of a primer, or covered with a top coat), there are a number of different methods for applying the therapeutic coating according to the present invention. These include dip coating and spray coating. Applicant's U.S. Pat. No. 6,821,549 issued Nov. 23, 2004 and U.S. Pat. No. 6,517,889 issued Feb. 11, 2003, both entitled “Process for Coating a Surface of a Stent”, discuss coating processes and disclose a novel method for coating a stent. The disclosures of these patent documents are incorporated herein by reference.

Another process for applying the therapeutic coating to an intravascular implant is as follows:

1. The implant is laser cut and then electropolished.

2. The electropolished implant is cleaned in a 1%-5% WN Potassium hydroxide or Sodium hydroxide for 1 hour. The temperature may be elevated to about 60 degrees Celsius to ensure proper cleaning. The cleaning can also be done with hexane or a solution of isopropyl alcohol.

3. The device is then washed with hot water. The washing may take place in a bath in which water is maintained at a constant temperature. Alternatively, the hot water is maintained on top of an ultrasonic bath so that the implant swirls as it is cleaned in the hot water.

4. The implant is dried at room temperature for up to 4 hours.

5. A primer is applied to the implant. The primer prepares the surface of the implant for the subsequent stages of bonding to the polymer.

6. Prepare functionalization chemicals. These chemicals could include hydride terminated polyphenyl_(dimethylhyrosiloxy) siloxanes; methylhydrosiloxane, phenylmethylsiloxane and methylhydrosiloxane-octylme-thylsiloxane copolymers, hydride terminated polydimethylsiloxanes, methylhydrosiloxanedimethylsiloxane copolymers; polymethylhyrosiloxanes, polyethylhydrosiloxanes. The chemicals could also include silanol functional siloxanes, like silanol terminated polydimethylsiloxanes; silanol terminated diphenylsiloxane-dimethylsiloxane copolymers; and silanol terminated polydiphenylsiloxanes. Suitable epoxy functional siloxanes include epoxy functional siloxanes include epoxypropoxypropyl terminated polydimethylsiloxanes and (epoxycyclohexylethyl) methylsiloxane-dimethylsiloxane copolymers.

7. The agents can be incorporated in the mixture of the polymer solution or can be bonded on to the surface of the polymer and also could be grafted on to the surface. One or more of the therapeutic agents is mixed with the coating polymers in a coating mixture. The therapeutic agent may be present as a liquid, a finely divided solid, or any other appropriate physical form. The mixture may include one or more additives, nontoxic auxiliary substances such as diluents, carriers, stabilizers etc. The best conditions are when the polymer and the drug have a common solvent. This provides a wet coating, which is a true solution.

8. The device is then placed in a mixture of functionalization chemicals for 2 hours at room temperature. An oscillating motion as described in the above-identified co-pending patent application can facilitate the coating process.

9. The device is then washed with methanol to remove any surface contaminants.

10. If there is a top coat of polymeric material that encapsulates the complete drug-polymer system, then the top coat is applied to the implant. The top coat can delay the release of the pharmaceutical agent, or it could be used as a matrix for the delivery of a different pharmaceutically active material.

11. The total thickness of the undercoat does not exceed 5 microns and the top coat is usually less than 2 microns.

In addition to applying a therapeutic coating to an intravascular implant, the implant can include an outer sheath where the sheath includes a therapeutic agent or agents to treat or prevent a disease process of a vascular disease of a patient. The intravascular implant may be, but is not limited to, a balloon catheter, stent, stent graft, stent preform, drug delivery catheter, atherectomy device, filter, scaffolding device, anastomotic clip, anastomotic bridge, suture material, metallic or non-metallic wire, embolic coil or a combination thereof.

Therapeutic Agents

The intravascular implants of the present invention may include one or more therapeutic substances. Each of the therapeutic agents mentioned herein may be placed in the reservoirs/tunnels alone or in any combination with each other. Two or more agents may be placed in the same reservoir, or multiple reservoirs may each include the same or different agents. The pharmaceutical agent can be an agent to treat or prevent the disease process of the vascular disease. The pharmaceutical agent may be imatinib mesylate, curcumin, sirolimus (rapamycin), or cyclosporin. The agent can include an anti-inflammatory agent, non-proliferative agent, anti-coagulant, anti-platelet agent, Tyrosine Kinase inhibitor, anti-infective agent, anti-tumor agent, anti-leukemic agent, or any combination thereof.

Examples of anti-inflammatory agents include, but are not limited to, Zinc compounds, dexamethasone and its derivatives, aspirin, non-steroidal anti-inflammatory drugs (NSAIDs) (such as ibuprofen and naproxin), TNF-α inhibitors (such as tenidap and rapamycin or derivatives thereof), or TNF-α antagonists (e.g., infliximab, OR1384), prednisone, dexamethasone, Enbrel®, cyclooxygenase inhibitors (i.e., COX-1 and/or COX-2 inhibitors such as Naproxen®, Celebrex®, or Vioxx®), CTLA4-Ig agonists/antagonists, CD40 ligand antagonists, other IMPDH inhibitors, such as mycophenolate (CellCept®), integrin antagonists, alpha-4 beta-7 integrin antagonists, cell adhesion inhibitors, interferon gamma antagonists, ICAM-1, prostaglandin synthesis inhibitors, budesonide, clofazimine, CNI-1493, CD4 antagonists (e.g., priliximab), p38 mitogen-activated protein kinase inhibitors, protein tyrosine kinase (PTK) inhibitors, IKK inhibitors, therapies for the treatment of irritable bowel syndrome (e.g., Zelmac® and Maxi-K® openers), or other NF-κB inhibitors, such as corticosteroids, calphostin, CSAIDs, 4-substituted imidazo[1,2-A]quinoxalines, glucocorticoids, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

Examples of anti-proliferative agents include, but are not limited to, cytochalasins, Taxol®, somatostatin, somatostatin analogs, N-ethylmaleimide, antisense oligonucleotides and the like, cytochalasin B, staurosporin, nucleotide analogs like purines and pyrimidines, Taxol®, topoisomerase inhibitor like topoisomerase I inhibitor or a topoisomerase II inhibitor, alkylating agents such as nitrogen mustards (mechlorethamine, cyclophosphamide, melphalan (L-sarcolysin)), nitrosoureas (carmustine (BCNU), lomustine (CCNU), semustine (methyl-CCNU), streptozocin, chlorozotocin), immunosuppressants (mycophenolic acid, thalidomide, desoxyspergualin, azasporine, leflunomide, mizoribine, azaspirane (SKF 105685)), paclitaxel, altretamine, busulfan, chlorambucil, cyclophosphamide, ifosfamide, mechlorethamine, melphalan, thiotepa, cladribine, fluorouracil, floxuridine, gemcitabine, thioguanine, pentostatin, methotrexate, 6-mercaptopurine, cytarabine, carmustine, lomustine, streptozotocin, carboplatin, cisplatin, oxaliplatin, iproplatin, tetraplatin, lobaplatin, JM216, JM335, fludarabine, aminoglutethimide, flutamide, goserelin, leuprolide, megestrol acetate, cyproterone acetate, tamoxifen, anastrozole, bicalutamide, dexamethasone, diethylstilbestrol, prednisone, bleomycin, dactinomycin, daunorubicin, doxirubicin, idarubicin, mitoxantrone, losoxantrone, mitomycin-c, plicamycin, paclitaxel, docetaxel, topotecan, irinotecan, 9-amino camptothecan, 9-nitro camptothecan, GS-211, etoposide, teniposide, vinblastine, vincristine, vinorelbine, procarbazine, asparaginase, pegaspargase, octreotide, estramustine, and hydroxyurea.

Examples of anti-coagulant agents include, but are not limited to, an RGD peptide-containing compound, heparin, antithrombin compounds, platelet receptor antagonists, anti-thrombin antibodies, anti-platelet receptor antibodies, aspirin, protaglandin inhibitors, platelet inhibitors, tick anti-platelet peptide, hirudin, hirulog, and warfarin.

Examples of anti-platelet agents include, but are not limited to, ReoPro®, ticlopidine, clopidrogel, and fibrinogen receptor antagonists.

Examples of Tyrosine Kinase inhibitors include, but are not limited to, c-Met, a receptor tyrosine kinase, and its ligand, scatter factor (SF), Epithelial Cell Kinase (ECK), inhibitors described in international patent applications WO 96/09294 and WO 98/13350 and U.S. Pat. No. 5,480,883 to Spada, et al., certain 2,3-dihydro-1H-[1,4]oxazino[3,2-g]quinolines, 3,4-dihydro-2H-[1,4]oxazino[2,3-g]quinolines, 2,3-dihydro-1H-[1,4]thiazino[3,2-g]quinolines, and 3,4-dihydro-2H-[1,4]thiazino[2,3-g]quinolines, EGF, PDGF, FGF, src tyrosine kinases, PYK2 (a newly discovered protein tyrosine kinase) and PTK-X (an undefined protein tyrosine kinase).

Examples of anti-infective agents include, but are not limited to Leucovorin, Zinc compounds, cyclosporins (e.g., cyclosporin A), CTLA4-Ig, antibodies such as anti-ICAM-3, anti-IL-2 receptor (Anti-Tac), anti-CD45RB, anti-CD2, anti-CD3 (OKT-3), anti-CD4, anti-CD80, anti-CD86, monoclonal antibody OKT3, agents blocking the interaction between CD40 and CD154 (a.k.a. “gp39”), such as antibodies specific for CD40 and/or CD154, fusion proteins constructed from CD40 and/or CD154/gp39 (e.g., CD40Ig and CD8gp39), β-lactams (e.g., penicillins, cephalosporins and carbopenams), β-lactam and lactamase inhibitors (e.g., augamentin), aminoglycosides (e.g., tobramycin and streptomycin), macrolides (e.g., erythromycin and azithromycin), quinolones (e.g., cipro and tequin), peptides and deptopeptides (e.g. vancomycin, synercid and daptomycin), metabolite-based anti-biotics (e.g., sulfonamides and trimethoprim), polyring systems (e.g., tetracyclins and rifampins), protein synthesis inhibitors (e.g., zyvox, chlorophenicol, clindamycin, etc.), nitro-class antibiotics (e.g., nitrofurans and nitroimidazoles), fungal cell wall inhibitors (e.g., candidas), azoles (e.g., fluoconazole and vericonazole), membrane disruptors (e.g., amphotericin B), nucleoside-based inhibitors, protease-based inhibitors, viral-assembly inhibitors, and other antiviral agents such as abacavir.

Examples of anti-tumor agents include, but are not limited to, DR3 Ligand (TNF-Gamma) and MIBG.

Examples of anti-leukemic agents include, but are not limited to, mda-7, human fibroblast interferon, mezerein, and Narcissus alkaloid (pretazettine).

Examples of chemotherapeutic agents include, but are not limited to, antibiotic derivatives (e.g., doxorubicin, bleomycin, daunorubicin, and dactinomycin), antiestrogens (e.g., tamoxifen), antimetabolites (e.g., fluorouracil, 5-FU, methotrexate, floxuridine, interferon alpha-2b, glutamic acid, plicamycin, mercaptopurine, and 6-thioguanine), cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate), hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone), nitrogen mustard derivatives (e.g., mephalen, chorambucil, mechlorethamine (nitrogen mustard) and thiotepa), steroids and combinations (e.g., bethamethasone sodium phosphate), and others (e.g., dicarbazine, asparaginase, mitotane, vincristine sulfate, vinblastine sulfate, and etoposide).

Examples of anti-angiogenic inhibitors include, but are not limited to, AG-3340 (Agouron, La Jolla, Calif.), BAY-12-9566 (Bayer, West Haven, Conn.), BMS-275291 (Bristol Myers Squibb, Princeton, N.J.), CGS-27032A (Novartis, East Hanover, N.J.), Marimastat (British Biotech, Oxford, UK), Metastat (Aeterna, St-Foy, Quebec), EMD-121974 (Merck KcgaA Darmstadt, Germany), Vitaxin (Ixsys, La Jolla, Calif./Medimmune, Gaithersburg, Md.), Angiozyme (Ribozyme, Boulder, Colo.), Anti-VEGF antibody (Genentech, S. San Francisco, Calif.), PTK-787/ZK-225846 (Novartis, Basel, Switzerland), SU-101 (Sugen, S. San Francisco, Calif.), SU-5416 (Sugen/Pharmacia Upjohn, Bridgewater, N.J.), SU-6668 (Sugen), IM-862 (Cytran, Kirkland, Wash.), Interferon-alpha, IL-12 (Roche, Nutley, N.J.), and Pentosan polysulfate (Georgetown University, Washington, D.C.).

Other therapeutic agents include thrombolytic agents such as tissue plasminogen activator, streptokinase, and urokinase plasminogen activators; lipid lowering agents such as antihypercholesterolemics (e.g. HMG CoA reductase inhibitors such as mevastatin, lovastatin, simvastatin, pravastatin, and fluvastatin, HMG CoA synthatase inhibitors, etc.); and anti-diabetic drugs, or other cardiovascular agents (loop diuretics, thiazide type diuretics, nitrates, aldosterone antagonistics (i.e. spironolactone and epoxymexlerenone), angiotensin converting enzyme (e.g. ACE) inhibitors, angiotensin II receptor antagonists, beta-blockers, antiarrythmics, anti-hypertension agents, and calcium channel blockers).

In an exemplary embodiment, the implants of the present invention, such as stents or stent preforms, include imatinib mesylate (GLEEVEC). GLEEVEC is a compound which is highly selective for PDGFR alpha, Beta-associated v-Ab1 tyrosine kinase. These compounds are not only able to inhibit acute vascular lesion formation after denudation injury, but also the development of chronic lesions such as those seen in diffused diseases in the vessel wall. GLEEVEC may be placed in the reservoirs of the stent or stent preform without any other agents. Alternatively, in combinatorial therapy, rapamycin may be combined with GLEEVEC. GLEEVEC can be combined with rapamycin standardization and delivered to the vessel wall via an intravascular implant.

As another example, heparin is known to dissolve clots in the vessel wall. By combining heparin with rapamycin, the stent is much less susceptible to clot formation.

In still another exemplary embodiment, the implants (e.g. stent or stent preform) may include curcumin (diferuloylmethane). Curcumin is an anti-inflammatory agent from curcuma longa, and it affects the proliferation of blood mononuclear cells and vascular smooth muscle cells. Curcumin independently inhibits the proliferation of rabbit vascular smooth muscle cells stimulated by fetal calf serum. Curcumin had a greater inhibitory effect on platelet derived growth factor stimulated proliferation than on serum-stimulated proliferation. Curcumin is very useful in the prevention of pathologic changes of atherosclerosis and restenosis. The possible mechanisms of the antiproliferative and apoptic effects of curcumin on vascular smooth muscle cells were studied in rat aortic smooth muscle cell line. Curcumin inhibits cell proliferation, arrested the cell cycle progression and induced cell apoptosis in vascular smooth muscle cells. Curcumin may be placed in the reservoirs of the stent or stent preform without any other agents. Alternatively, in combinatorial therapy, curcumin may be combined with another therapeutic agent.

Additional pharmaceutical agents as well as methods to apply these agents are set forth in U.S. Pat. No. 6,585,764 to Wright et al, as well as, commonly owned U.S. patent application Ser. No. 10/696,174 entitled “Rationally Designed Therapeutic Intravascular Implant Coating” and are herein incorporated by reference.

Clinical Experiments

Materials and Methods—Spindle-shaped and rhomboid smooth muscle cells (S-SMCs and R-SMCs, respectively) were isolated from the left anterior descending coronary artery media of 8-month-old domestic crossbred pigs. S-SMCs were isolated by enzymatic digestion and R-SMCs by tissue explantation (Hao et al., ATVB 22:1093-1099, 2002). SMCs were cultured in Dulbecco's modified Eagle medium (DMEM; Gibco BRL, Paisley, UK) containing 10% fetal calf serum (FCS; Amimed; France) and were used from passage 9 to 14.

Imatinib was diluted in DMSO. The first set of experiments was set up to find the concentration to use for the best effect of each drug on SMCs. Tested dilutions were as folow: 0.001, 0.01, 0.1, 1 and 10 μg/ml.

Curcumin were diluted in DMEM. Tested dilutions were as follow: 0.1, 1, 1.25, 2.5, 5 and 10 μg/ml.

Sirolimus was diluted in DMSO. Cyclosporin was diluted in EtOH. They were used simultaneously at a concentration of 10 and 100 nM.

Controls consisted of SMCs incubated in DMEM with 10% FCS or SMCs incubated in DMEM with 10% FCS and DMSO or EtOH.

Cells were plated at a density of 10'000 cells/cm2 in DMEM+10% FCS. Twenty-four hours after plating, medium was incubated with drugs or vehicle in DMEM+10% FCS. Medium was changed every 2 (for sirolimus and cyclosporin) or 3 (for imatinib and curcumin) days and cells were harvested at 6 days of treatment. Cells were counted using a hemocytometer. Cell viability was evaluated by Trypan blue exclusion test. Results were expressed as a mean percentage of control conditions.

For evaluation of migratory capacity, R-SMCs were plated at a density of 10'000 cells/cm² in DMEM in the presence of 10% FCS. At confluence, R-SMCs were scratched with a silicon coated stick to obtain a 0.8 mm-wide in vitro wound (Bochaton-Piallat et al., ATVB 16: 815-820, 1996) and photographed in phase contrast using a Zeiss Axiovert 35 photomicroscope (Carl Zeiss, Jena, Germany). Fresh medium plus FCS alone or containing one of the above-described agents was added. After 18 hours, nuclear staining using propidium iodide (0.05 mg/ml, Fluka) was performed and migrating cells invading the empty space were counted using a Zeiss Axiovert photomicroscope (Carl Zeiss) and a Metamorph interactive image-analysis system (Universal Imaging Corporation, Downingtown, Pa., USA). Six randomly pre-selected fields (length, 2.5 mm) were analyzed per condition. Results were calculated as the total number of migrated cells per field and expressed as percentage of control conditions.

For SDS-PAGE, samples were resuspended in 0.4 M Tris HCl, pH=6.8, containing 1% SDS, 1% dithiothreitol, 1 mM phenylmethyl sulfonyl fluoride, 1 mM Nα-p-tosyl-L-arginine methyl ester and boiled 3 minutes. Protein content was determined according to Bradford (Bradford, Anal Biochem 72:248-254, 1976). Fifteen μg proteins were electrophoresed on a 12% gel and stained with Coomassie Blue.

Western-blotting was performed using a mouse monoclonal IgG2a specific for α-smooth muscle (SM) actin (Skalli, J cell Biol 103:2787-2796, 1986) and a rabbit polyclonal IgG specific for S100A4 (Dako, Glostrup, Denmark). For the detection of α-SM actin 0.25 to 1 μg of protein were loaded in 12% gradient gels followed by electrophoresis. The amount of protein loaded for S100A4 detection were 15 μg. Separated proteins were transferred to nitrocellulose filters which were incubated with anti α-SM actin (1:500) or anti-S100A4 (1:1500) antibodies for 2 hours. After three rinses, a second incubation for 1 hour was performed with goat anti-mouse or anti-rabbit IgG labeled with peroxidase. Enhanced chemiluminescence was used for detection (Amersham, Buckinghamshire, England). Modification of α-SM actin and S100A4 expression was evaluated by densitometric scanning of western-blots using a computerized scanner (Arcus II, AGFA, Mortsel, Belgium) and expressed as a mean percentage of control conditions.

Results—Imatinib and curcumin doses showing the most powerful effect on SMC proliferation were: 0.001 and 0.01 μg/ml for imatinib and 2.5 and 5 μg/ml for curcumin. The other tested doses showed either a toxic or no effect.

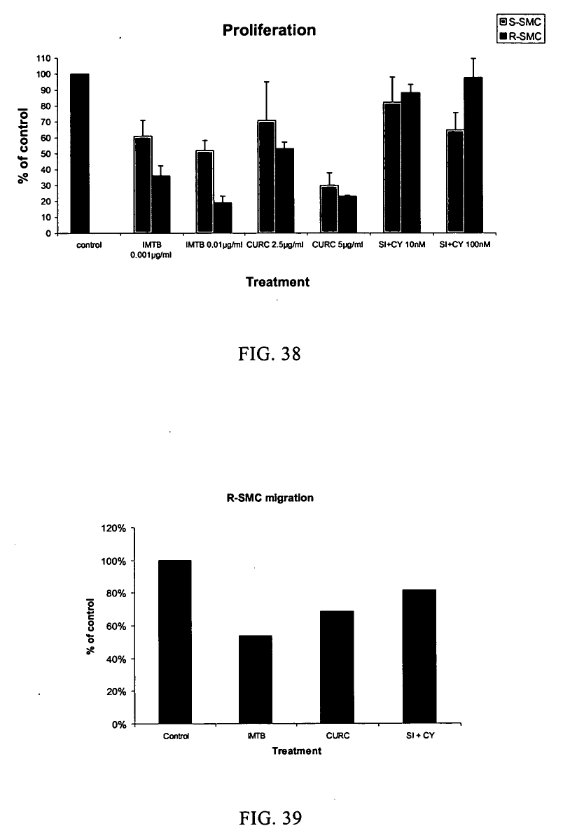

Compared with controls, proliferation of S-SMCs was decreased with imatinib at 0.01 μg/ml (p<0.05) whereas that of R-SMCs was reduced at 0.001 and 0.01 μg/ml (p<0.001 in both cases) (FIG. 38). Curcumin decreased S-SMC proliferation at 5 μg/ml (p<0.01) and R-SMC proliferation at 2.5 (p<0.01) and 5 μg/ml (p<0.001). Sirolimus+cyclosporin treatment slightly decreased S-SMC proliferation at 100 nM and did not affect R-SMC proliferation. The number of dead cells was negligible in all cases (<1% in all conditions studied).

FIG. 38 shows the effect of imatinib, curcumin, sirolimus and cyclosporin on S- and R-SMC proliferation. The results (mean±SEM, n=3 to 5) are given as % of control conditions i.e. cells treated with vehicle (IMTB=Imatinib, CURC=Curcumin, SI=sirolimus, CY=cyclosporine).

A preliminary migration assay was performed using R-SMCs because of their high migratory capacity. The highest efficient tested doses for each drug were used: 0.1 μg/ml for imatinib, 51 g/ml for curcumin and 100 nM for sirolimus and cyclosporin (FIG. 39). Imatinib and curcumin markedly reduced migration of R-SMCs compared with control conditions; imatinib acted to a greater extent than curcumin. Cyclosporin and rapamycin did not show marked effect on R-SMC migration.

FIG. 39 shows the effect of imatinib, curcumin, sirolimus and cyclosporin on R-SMC migration. The results are given as % of control conditions i.e. cells treated with vehicle (IMTB=Imatinib, CURC=Curcumin, SI=sirolimus, CY=cyclosporine).

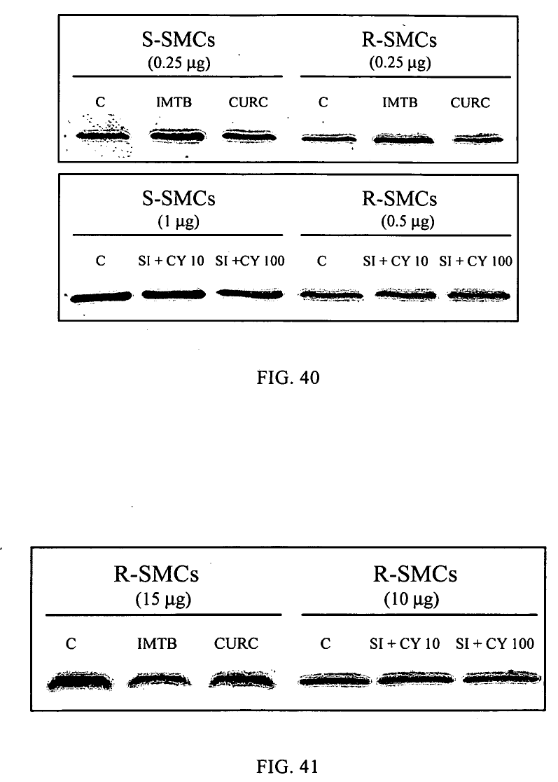

The expression of α-SM actin, a well accepted SMC differentiation marker, was evaluated by western-blotting (FIG. 40). Densitometric scanning of western-blots (Table 1) showed that imatinib (0.01 μg/ml) slightly increased α-SM actin expression in both cell types compared with control conditions. Curcumin (5 μg/ml) did not affect α-SM actin expression. These results need to be confirmed by additional experiments. When treated with sirolimus+cyclosporin at 100 nM α-SM actin expression of S-SMCs was significantly decreased whereas that of R-SMCs was not affected.

FIG. 40 shows the effect of imatinib, curcumin, sirolimus and cyclosporin on α-SM actin expression in S- and R-SMCs. (Representative gel; C=control i.e. cells treated with vehicle, IMTB=Imatinib, CURC=Curcumin, SI=sirolimus, CY=cyclosporine).

TABLE 1

| Condition | S-SMC | R-SMC |
|---|---|---|
| Control | 100 | 100 |
| IMTB 0.01 μg/ml | 131 ± 6 (n = 2) | 143 ± 31 (n = 3) |
| CURC 5 μg/ml | 102 ± 1 (n = 2) | 99 ± 12 (n = 3) |
| SI + CY 10 nM | 82 ± 12 (n = 3) | 132 ± 15 (n = 3) |
| SI + CY 100 nM | 77 ± 5* (n = 4) | 116 ± 17 (n = 5) |

Table 1 shows the effect of imatinib, curcumin, sirolimus and cyclosporin on α-SM actin expression in S- and R-SMCs. The results are densitometric scanning of western-blots expressed as % of control i.e. cells treated with vehicle (mean±SEM, n=number of experiments, *p≦0.01 compared with control).

S100A4, a newly identified protein in our laboratory as a selective marker of R-SMCs, was evaluated by western-blotting in both SMC phenotypes (FIG. 41). We confirm that S100A4 is strongly expressed in R-SMCs and not detectable in S-SMCs (Brisset et al., manuscript in preparation). Densitometric scanning of western-blots (Table 2) showed that imatinib and curcumin slightly decreased S100A4 expression in R-SMCs. This result needs to be confirmed by additional experiments. When used at the highest dose, Sirolimus+cyclosporin treatment significantly decreased the expression of S100A4 in R-SMCs.

FIG. 41 shows the effect of sirolimus and cyclosporin on S100A4 expression in R-SMCs. (Representative gel; C=control i.e. cells treated with vehicle, SI=sirolimus, CY=cyclosporine).

TABLE 2

| Condition | S-SMC | R-SMC |
|---|---|---|
| Control | not detectable | 100 |
| IMTB 0.01 μg/ml | not detectable | 66 ± 16 (n = 3) |
| CURC 5 μg/ml | not detectable | 85 ± 8 (n = 3) |
| SI + CY 10 nM | not detectable | 82 ± 13 (n = 4) |
| SI + CY 100 nM | not detectable | 76 ± 18* (n = 6) |

Table 2 shows the effect of imatinib, curcumin, sirolimus and cyclosporin on S100A4 expression in S- and R-SMCs. Results are densitometric scanning of western-blots expressed as % of control i.e. cells treated with vehicle (mean±SEM, n=number of experiments, *p≦0.05 compared with control).

Conclusions—It should be also noted that the doses used for all tested drugs do not cause cell death in both cell types.

Imatinib significantly decreases S- and R-SMC proliferation; its effect is more important on R-SMCs compared with S-SMCs. It also markedly reduces R-SMC migration. Immunoblotting studies show that imatinib slightly increases α-SM actin expression in both cell types; this is associated to a slight decrease of S100A4 in R-SMCs. It should be noted that S100A4 is not detectable in S-SMCs. Curcumin exhibits the same effect on proliferation as imatinib; however curcumin acts on migratory activity to a lesser extent than imatinib. It has no effect on SMC differentiation level. Whereas proliferation studies are statistically well determined, the effects of these 2 drugs on SMC migration and phenotypic markers remain to be clearly established. In order to reach statistically valuable results, we propose to repeat migratory activity assays and immunoblot experiments at least twice on each SMC phenotype. Nevertheless, the results indicate that imatinib and curcumin are very efficient in order to reduce proliferation and migration of porcine coronary artery SMCs and suggest that R-SMCs are more sensitive to these drugs compared with S-SMCs. The preliminary results on α-SM actin and S100A4 expression suggest that the SMC phenotypes are slightly modulated by these treatments but this remains to be confirmed.

Sirolimus+cyclosporin treatment decreases the proliferation of S-SMCs only at the dose of 100 nM. This effect is accompanied by a slight decrease in the expression of α-SM actin. As mentioned above, S100A4 is not detectable in S-SMCs. In R-SMCs, proliferative and migratory activity as well as α-SM actin expression are not affected by the treatment. However, at 100 nM, the expression of S100A4 is decreased. Therefore sirolimus+cyclosporin treatment when used at the highest doses acts differently on the two SMC phenotypes. It modulates the phenotype of S-SMCs towards less differentiated features (decrease of α-SM actin) and the phenotype of R-SMCs towards more differentiated features (decrease of S100A4). The results that R-SMCs proliferation and migration (to be confirmed) are not affected by these treatments are surprising.

Previous work has extensively shown that α-SM actin represents a very useful SMC differentiation marker. S100A4 can be used as a new marker of atheroma-prone SMC phenotype applicable to human situations. These two markers should be efficient in the evaluation of pharmacologic activities of different drugs influencing SMCs. Imatinib and curcumin exert powerful inhibitory actions on SMC activation. Imatinib in particular appears to be very efficient in order to produce SMC stabilization and differentiation.

Rationally Designed Implant

In a related invention, the implants of the present invention (e.g. structurally variable stents and stent preforms) may be tailored to treat or prevent a disease process of a vascular disease. That is, the selected therapeutic agent(s) of the implant is based on the genesis of the disease and the underlying morphology of the disease. This concept evolved from the need to identify key events in the molecular pathology of fibroproliferative restenotic disease in order to develop specific and effective treatments. Restenosis is no longer just identified as a hyperproliferative disease, but more specifically it is viewed as a fibroproliferative disease with well defined pathologic cascade of events and interactions.

Therefore, therapeutic agents to be delivered via a stent, stent preform, and/or an implant coating into the vascular vessel wall are designed to treat or prevent prevalent/existing disease processes of a patient that create the problem. The disease processes can include, but are not limited to, acute myocardial infarction, thrombotic lesions, unstable angina, fibrotic disease, total occlusion, hyperproliferative vascular disease, vulnerable plaque, and diabetic vascular diffused disease.

Techniques used to identify these events or processes include an angiogram, fluoroscopy, CT scan, MRI, intravascular MRI, lesion temperature determination, genetic determination, etc. An angiogram is acquired by injecting a radiopaque dye into the vascular system, usually by means of a catheter. The radiopaque dye infuses the vessels, and a radiological projection is made of the infused vessels onto a radiographic sensor. The resultant angiogram will reveal the lumens of the vessels as the radiopaque dye flows through them. A narrowing of the infused lumen will provide an indication of an obstruction of a vessel and a potential condition for infarction.

Fluoroscopy generates images of internal structures on a video monitor during energization of an x-ray tube. Fluoroscopy may use x-ray to produce real-time video images. After the x-rays pass through the patient, they are captured by a device called an image intensifier and converted into light. The light is then captured by a TV camera and displayed on a video monitor.

A CT scan (computed tomography scan) is a special radiographic technique that uses a computer to assimilate multiple X-ray images into a 2 dimensional cross-sectional image. This can reveal many soft tissue structures not shown by conventional radiography. Scans may also be dynamic in which a movement of a dye is tracked. A special dye material may be injected into the patient's vessel prior to the scan to help differentiate abnormal tissue and the vasculature.

An MRI scan (magnetic resonance imaging scan) is a method of visualizing soft tissues of the body by applying an external magnetic field that makes it possible to distinguish between hydrogen atoms in different environments. The images are very clear and are particularly good for the brain, spinal cord, joints, abdomen and soft tissue. Intravascular MRI uses an MR probe which may be built into catheters, allowing diagnostically useful high resolution images to be obtained from within small, intravascular structures.

Identifying lesion temperature is a technique without significant clinical experience. The temperature of a lesion is measured to determine whether it is unstable or not. A catheter, probe, or the like, is inserted into the vasculature near the lesion, and the temperature of the lesion may be measured. For example, one technique is measuring lesion temperature by analyzing stress patterns in a lesion molding balloon which are revealed under a polariscope after the balloon has been molded to the lesion and then removed from the body for inspection. In another example, a balloon coating which changes color in accordance with a temperature experience may be used. Also, temperature of lesion may be measured using an infrared sensor.

Finally, genetic determination is a technique to identify differently expressed genes in the process of a vascular disease. The systematic and comprehensive characterization of gene transcription is possible using whole genome sequencing, bioinformatics and high throughput transcription profiling technologies. Based on specifically identified genes in a vascular disease, a disease process can be identified, and the vascular disease may be treated or prevented.

Given the identification of the prevalent/existing processes of restenosis, construction of a tailored implant can be designed which may be used to treat or prevent processes of restenosis from people with various risk factors and underlying mechanisms. That is, restenosis is different in every individual depending on the underlying conditions that constitute the vascular disease. Each individual may have different disease processes which can be identified and treated with a rationally designed implant. The implant may deliver a therapeutic agent locally while systemically the same or other therapeutic agents may be delivered. The combination of local and systemic drug delivery treats or prevents one or more disease processes.

To make a therapeutic intravascular implant to treat or prevent a specific disease process of a vascular disease, the disease process or processes which are prevalent in the vessel wall of the patient are identified. This identification can be achieved using a technique or a combination of techniques previously mentioned. A therapeutic agent or combination of agents is selected for treating or preventing the identified disease process or processes. The intravascular implant includes a therapeutically effective amount of a first agent to treat or prevent the disease process.

One way to identify a disease process in the vessel wall of the patient and to treat the vascular disease is to perform more than one procedure on the patient. First, a preliminary procedure may be performed with the goal of determining the prevalent disease process. Based on the identification of the disease process, an implant may be coated with at least one therapeutic agent, or a pre-coated implant having the desired therapeutic agent or agents may be obtained. Then, the patient may undergo a second procedure for implanting the coated implant in the patient's vasculature. Alternatively, a single procedure may be performed to identify the disease process and insert the coated implant in the patient. In this regard, it is envisioned that the implant could be coated with the desired agent or agents at the site of the procedure (i.e. in or near the operating room), or a pre-coated implant having the desired therapeutic agent or agents may be selected from an inventory of pre-coated implants.

### EXEMPLARY EMBODIMENTS

### Example 1

### Curcumin-Eluting Implant

The following examples describe various embodiments of the present invention. It should be understood that these examples do not limit the inventive implants disclosed herein. In one exemplary embodiment, the implant includes curcumin as the only therapeutic agent. The implant may be a stent or a stent preform as previously described. The implant includes reservoirs and/or tunnels configured for carrying the curcumin. The curcumin is placed on the surface of the implant and within the reservoirs or is placed only in the reservoirs. Placement of the curcumin on the stent and/or in the reservoirs is performed may adding the curcumin to a solvent, adding the solvent-curcumin to the stent, and allowing the solvent to dissipate. The solvent may be DMSO, DMEM, EtOH, or other suitable solvent. The implant may optionally include a top coat placed over the curcumin on the stent. The top coat may be bioerodible to control the release of the curcumin. The implant of this example, and its derivatives, is free from any polymeric material. That is, no polymer is used to make the implant, and the completed implant, ready to be inserted in a patient, is free of any polymer.

### Example 2

### Imatinib Mesylate-Eluting Implant

In another exemplary embodiment, the implant includes imatinib mesylate (GLEEVEC) as the only therapeutic agent. The implant may be a stent or a stent preform as previously described. The implant includes reservoirs and/or tunnels configured for carrying the imatinib. The imatinib is placed on the surface of the implant and within the reservoirs or is placed only in the reservoirs. Placement of the imatinib on the stent and/or in the reservoirs is performed may adding the imatinib to a solvent, adding the solvent-imatinib to the stent, and allowing the solvent to dissipate. The solvent may be DMSO, DMEM, EtOH, or other suitable solvent. The implant may optionally include a top coat placed over the imatinib on the stent. The top coat may be bioerodible to control the release of the imatinib. The implant of this example, and its derivatives, is free from any polymeric material. That is, no polymer is used to make the implant, and the completed implant, ready to be inserted in a patient, is free of any polymer.

### Example 3

### Combinational Therapeutic Implant: Curcumin+Rapamycin

In another exemplary embodiment, the implant includes curcumin and rapamycin. The implant may be a stent or a stent preform as previously described. The implant includes reservoirs and/or tunnels configured for carrying the curcumin and/or rapamycin. The two therapeutic agents may be placed on the implant as follows. The rapamycin may be placed within the reservoirs only, and the curcumin may be placed on the rapamycin, or vice versa. The rapamycin may be placed within the reservoirs only, and the curcumin may be placed on the surface of the implant, or vice versa. The rapamycin may be placed within the reservoirs and the surface of the implant, and the curcumin may be placed over the rapamycin, or vice versa. The rapamycin may be placed within the reservoirs and on the surface of the implant, and the curcumin may be placed on the surface rapamycin or on the reservoir rapamycin, or vice versa. A bioerodible barrier coat may be placed between the rapamycin and curcumin to separate the agents. The implant may optionally include a top coat placed over the agents on the stent. The top coat may be bioerodible to control the release of the agents. The implant of this example, and its derivatives, is free from any polymeric material. That is, no polymer is used to make the implant, and the completed implant, ready to be inserted in a patient, is free of any polymer.

### Example 4

### Combinational Therapeutic Implant: Imatinib Mesylate+Rapamycin

In another exemplary embodiment, the implant includes imatinib mesylate and rapamycin. The implant may be a stent or a stent preform as previously described. The implant includes reservoirs and/or tunnels configured for carrying the imatinib and/or rapamycin. The two therapeutic agents may be placed on the implant as follows. The rapamycin may be placed within the reservoirs only, and the imatinib may be placed on the rapamycin, or vice versa. The rapamycin may be placed within the reservoirs only, and the imatinib may be placed on the surface of the implant, or vice versa. The rapamycin may be placed within the reservoirs and the surface of the implant, and the imatinib may be placed over the rapamycin, or vice versa. The rapamycin may be placed within the reservoirs and on the surface of the implant, and the imatinib may be placed on the surface rapamycin or on the reservoir rapamycin, or vice versa. A bioerodible barrier coat may be placed between the rapamycin and imatinib to separate the agents. The implant may optionally include a top coat placed over the agents on the stent. The top coat may be bioerodible to control the release of the agents. The implant of this example, and its derivatives, is free from any polymeric material. That is, no polymer is used to make the implant, and the completed implant, ready to be inserted in a patient, is free of any polymer.

All references cited herein are expressly incorporated by reference in their entirety.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention.

What is claimed is:

1. A stent comprising:
a tubular body;
a plurality of reservoirs disposed in the tubular body; and
a therapeutic agent disposed in the reservoirs,
wherein the stent is free of polymeric material.

2. The stent of claim 1, wherein the therapeutic agent is curcumin.

3. The stent of claim 1, wherein the therapeutic agent is imatinib mesylate.

4. The stent of claim 1, further including a top coat covering the therapeutic agent.

5. The stent of claim 1, wherein at least one of the reservoirs is a concave dome-shaped indentation disposed in a surface of the tubular body.

6. The stent of claim 5, wherein the reservoirs are disposed on an outer surface of the tubular body, the outer surface being positionable against a vessel wall.

7. The stent of claim 6, further including a top coat covering the therapeutic agent.

8. The stent of claim 7, wherein the top coat is bioerodible.

9. The stent of claim 1, wherein the therapeutic agent is disposed in the reservoirs and on a surface portion of the tubular body.

10. The stent of claim 1, wherein the tubular body includes a longitudinal cylindrical base structure including a first end portion, a second end portion, a mid-portion interposed between the first and second end portions, and a plurality of linear strut members connecting the mid-portion to the first and second end portions, the first and second end portions having a first pattern and the mid portion having a second pattern different from the first pattern, the second pattern including a plurality of articulations.

11. The stent of claim 10, wherein the therapeutic agent is curcumin.

12. The stent of claim 11, further including a top coat covering the curcumin.

13. The stent of claim 10, wherein the therapeutic agent is imatinib mesylate.

14. The stent of claim 13, further including a top coat covering the imatinib.

15. The stent of claim 14, wherein the top coat is bioerodible.

16. The stent of claim 10, wherein the therapeutic agent is disposed in the reservoirs and on a surface portion of the tubular body.

17. A drug-eluting stent made from the process of:
providing a polymer-free stent body having a plurality of reservoirs disposed therein;
diluting a therapeutic agent in a polymer-free solvent to form an agent-solvent mixture;
coating the stent with the agent-solvent mixture; and
allowing the solvent to dissipate from the stent thereby leaving the agent disposed on the stent.

18. The stent of claim 17, wherein the therapeutic agent is curcumin, imatinib, or a combination thereof.

19. The stent of claim 18, further including placing a top coat over the agent disposed on the stent.

20. The stent of claim 19, wherein the top coat is bioerodible.
